(19) United States
(12) Patent Application Publication
(10) Pub. No.: US 2015/0219652 A1
(43) Pub. Date: Aug. 6, 2015

(54) BINDING-INDUCED FORMATION OF DNA THREE-WAY JUNCTIONS FROM NON-DNA TARGETS

(71) Applicants: Xiao Chun Le, Edmonton (CA); Feng Li, Edmonton (CA)

(72) Inventors: Xiao Chun Le, Edmonton (CA); Feng Li, Edmonton (CA)

(73) Assignee: THE GOVERNORS OF THE UNIVERSITY OF ALBERTA, Edmonton (CA)

(21) Appl. No.: 14/556,853

(22) Filed: Dec. 1, 2014

Related U.S. Application Data
(60) Provisional application No. 61/910,254, filed on Nov. 29, 2013.

(51) Int. Cl. G01N 33/573

(52) U.S. Cl. CPC G01N 33/573; G01N 2333/96441; G01N 2333/974

(57) ABSTRACT

A method of detecting a non-DNA target includes the use of a first nucleic acid motif linked to a first affinity ligand which binds specifically to the target and having a first toehold domain and a first binding domain, and a second nucleic acid motif linked to a second affinity ligand which binds specifically to the target and having a second binding domain, wherein the first and second binding domains are complementary to each other. Upon contact with the target, the first and second nucleic acid motifs bind to form a target-ligand complex. The formation of the complex causes displacement of an output nucleic acid motif. This method may be used with detectable beacons in an imaging or diagnostic method, and particularly in a point of care diagnostic method.

### CROSS-REFERENCE TO RELATED APPLICATION

This application claims the priority benefit of U.S. Provisional Patent Application No. 61/910,254, filed on Nov. 29, 2013, entitled “Binding-Induced Formation of DNA Three-Way Junctions”, the entire contents of which are incorporated herein by reference.

### FIELD OF THE INVENTION

The present invention relates to protein-responsive DNA devices and assemblies.

### BACKGROUND

DNA three-way junctions (DNA-TWJs) are important building blocks to construct DNA architectures and dynamic assemblies. Target-responsive DNA TWJs can also be designed into DNA devices for molecular diagnostic, sensing, and imaging applications. While successful TWJs have been focused on DNA, the benefits have not been extended to proteins and other targets which do not possess the base-pairing properties of DNA.

### SUMMARY OF THE INVENTION

In one aspect, the invention may comprise a method of detecting a non-DNA target, comprising the steps of:

(a) providing a first nucleic acid motif linked to a first affinity ligand which binds specifically to the target, and a second nucleic acid motif linked to a second affinity ligand which binds specifically to the target;

(b) wherein the first nucleic acid motif has a first toehold domain linked to a first binding domain complementary to the second nucleic acid motif;

(c) contacting the target with the first and second nucleic acid motifs to form a target-ligand complex wherein the first and second affinity ligands are bound to the target, and the first nucleic acid motif is hybridized to the second nucleic acid motif, wherein the formation of the complex causes displacement of an output nucleic acid motif.

In one embodiment, the target may be a protein. In one embodiment, each of the first, second and output nucleic acid motifs comprise DNA. In one embodiment, the output DNA motif is hybridized to one of the first or second DNA motifs, and is displaced by the formation of the complex.

Preferably, the method is implemented without the use of enzymes and/or thermal cycling.

In another embodiment, the method further comprises the step of contacting the target-ligand complex with a detection probe comprising the output DNA motif. In one embodiment, the detection probe comprises a second toehold domain complementary to the first toehold domain, and a displacement domain complementary to a displacement domain of the second DNA motif, wherein hybridization of the detection probe to the target-ligand complex displaces the output DNA motif.

In one embodiment, the first and second affinity ligands are the same or different, and at least one is an antibody or an aptamer.

In one embodiment, the method further comprises the use of a displacement beacon which provides a detectable signal upon displacement of the output DNA motif. The displacement beacon may comprise a fluorophore carried on the detection probe. The detection probe comprises a fluorophore and a quencher, wherein the quencher is linked to the third DNA motif. The quencher may comprise a dark quencher.

In one embodiment, the output DNA motif is used in a catalytic DNA circuit and/or a dynamic DNA assembly method.

Embodiments of the invention may be used to detect an antigen in a biological sample or on the surface of a cell, and/or may be used to operate as an imaging method, a diagnostic method, or a point-of-care diagnostic method.

In another aspect, the invention may comprise a protein-DNA three way junction complex comprising a first DNA motif linked to a first affinity ligand bound specifically to the protein, a second DNA motif linked to a second affinity ligand bound specifically to the protein, wherein the first and second DNA motifs comprise domains hybridized to each other, and a third DNA motif hybridized to the first and second DNA motifs.

In one embodiment, the protein-DNA complex further comprises a detectable beacon, which may comprise a fluorophore.

In another aspect, the invention may comprise a kit for detecting a protein, comprising a first nucleic acid motif linked to a first affinity ligand which binds specifically to the protein and having a first toehold domain and a first binding domain, and a second nucleic acid motif linked to a second affinity ligand which binds specifically to the protein and having a second binding domain, wherein the first and second binding domains are complementary to each other, and a output nucleic acid motif which is displaced by the binding of the first and second nucleic acid motifs to the protein and to each other.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention,

FIG. 1. Detection of prostate specific antigen (PSA) using binding-induced TWJ. (A) Schematic showing the design of PSA-responsive TWJ. (B) Real-time monitoring of the fluorescence increases over time from the determination of varying concentrations (0-285 ng/mL) of PSA. (C) Increases in fluorescence signals as a function of concentrations of PSA in buffer (red line) and in 10-time diluted human serum (blue line). Fluorescence measurements were taken at 60 min.

FIG. 2. Detection of human α-thrombin using binding-induced TWJ. (A) Schematic showing the design of thrombin-responsive TWJ. (B) Increases in fluorescence signals as a function of concentrations of thrombin in buffer. Fluorescence measurements were taken at 60 min.

FIG. 3. Detection of human α-thrombin using binding-induced TWJ, (A) Schematic showing the design of thrombin-responsive TWJ. This embodiment provides an example of tuning the kinetics from the binding part. (B) Increases in fluorescence signals over time. (C) Increases in fluorescence signals over time. Both (B) and (C) illustrate that by tuning the binding part, the signal can be generated instantly upon binding of the target.

FIG. 4. Characterization of the oligonucleotides involved in the formation of binding-induced TWJ and strand displacement. (A) Schematic showing that binding of the two probes to streptavidin triggers the formation of TWJ and the release of fluorescent oligo C. Provides an embodiment of the formation of TWJ from the designed motifs. (B) Native PAGE analysis followed by SYBR Gold staining. (C) Native PAGE analysis without using subsequent staining. Fluorescence images were due to the FAM label on the oligos. Lane 1 contained 2 μM B*C. Lane 2 contained 2 μM TB. Lane 3 contained 1 μM T*C*:C and 1 μM T*C*. Lane 4 was from the analysis of a mixture containing 2 μM B*C, 2 μM TB, 1 μM T*C*:C, and 1 μM T*C*. Lane 5 was from the analysis of a mixture containing 2 μM B*C, 2 μM TB, 1 μM T*C*:C, 1 μM T*C*, and 1 μM streptavidin.

FIG. 5. (A) Schematic showing the design for real-time monitoring of the formation of binding-induced TWJ. DNA motif T*C* was labeled with a fluorephore and motif C was labeled with a quencher. The fluorescently labeled T*C* was initially hybridized with C, thus its fluorescence was quenched by the quencher. Binding of the two biotinylated DNA motifs TB and B*C to the same target streptavidin triggered the formation of TB:B*C:T*C* TWJ and simultaneous release of the quencher-labeled C, turning on the fluorescence. (B and C). Optimizing the kinetics of binding-induced TWJ by using different designs of motif TB in the presence of 10 nM streptavidin (B) and in the absence of streptavidin (C). Kinetics can be tuned from either toehold part (red region) or binding part (green region). The length of the toehold domain T (m) was varied from 6 nt to 15 nt, and the length of domain B (n) was fixed at 6 nt. The positive control (P.C.) contained 10 nM probe T8C20 and 20 nM T*C*:C in TE-Mg buffer. The negative control (N. C.) contained only 20 nM T*C*:C in TE-Mg buffer.

FIG. 6. Effect of incubation temperature (37° C. and 25° C.) on the formation kinetics of binding-induced TWJ. The reaction mixture for the streptavidin sample contained 20 nM probe T*C*:C, 20 nM probe TB, 20 nM probe B*C, 10 nM target streptavidin, 50 nM ROX, 1 μM polyT oligo, and TE-Mg buffer. In the blank, all reagents were the same as in the sample solution, except that there was no streptavidin added.

FIG. 7. Comparing four different DNA strand-displacement strategies and their kinetic profiles, (A) The four available strand-displacement strategies, [15-28] including toehold-mediated DNA strand displacement (a—prior art), binding-induced TWJ (b), DNA strand displacement mediated by associative DNA toehold (c—prior art), and binding-induced DNA strand displacement (d). (B) Kinetic profiles from the determination of 10 nM target using the four strand-displacement strategies. (C) Background fluorescence observed from comparing the four strand-displacement strategies. The observed rate constant kobs was 3.31×10−3 s⁻¹ for (a), 0.61×10−3 s⁻¹ for (b), 0.16×10−3 s⁻¹ for (c), and 0.06×10−3 s⁻¹ for (d).

FIG. 8 illustrates the binding-inducted DNA TWJ making use of two DNA motifs, each conjugated to an affinity ligand. The binding of two affinity ligands to the target molecule triggers assembly of the DNA motifs and initiates the subsequent DNA strand displacement, resulting in a binding-induced TWJ. Real-time fluorescence monitoring of the binding-induced TWJ enables detection of specific protein targets. This figure provides an illustration of an embodiment of the invention, comprising a displacement beacon for detection. In the example, two strands of DNA in the beacon are conjugated with a fluorescence donor and a fluorescence acceptor.

FIG. 9 is a schematic showing the general principle of the binding-induced formation of DNA three-way junction (TWJ). Binding of the target molecule to the two specific affinity ligands brings two DNA motifs TB and B*C to close proximity, forming the TB:B*C duplex. The formation of TB:B*C triggers a subsequent strand displacement between TB:B*C and C, resulting in a stable binding-induced TWJ (TB:B*C:T*C*) and the release of C. The output C can be detected with any nucleic acid methods. Symbols in this scheme do not reflect the actual sizes of the molecules.

FIGS. 10A and B illustrate one embodiment of the invention. (FIG. 10A). Hairpin DNA and enzyme-free circuit. (FIG. 10B). Quantification of PSA in buffer; PSA detected using Hairpin DNA and enzyme-free circuit technique. Concentration range of 0-2 nM PSA, detection limit 16 pM.

FIG. 11 illustrates an embodiment of the hairpin DNA and hybridization chain reaction scheme.

FIG. 12 illustrates an embodiment of the invention. (A) Circular DNA and rolling circle amplification scheme.

FIG. 13 illustrates an embodiment of the invention. (A) Human alpha-thrombin detection scheme. (B) Increases in fluorescence signals as a function of concentrations of thrombin in buffer.

FIG. 14 illustrates real-time detection of a secreted target from a target cell scheme. Using a beacon in the solution, secreted molecules from targeted cells are detected.

FIG. 15 illustrates prostate specific antigen detection on a magnetic bead scheme.

FIG. 16 illustrates one embodiment of a cell imaging application of the disclosure, using a single marker. Using a beacon in the solution, cell surface molecules of targeted cells are detected.

FIG. 17 illustrates one embodiment of a cell imaging application of the disclosure, using co-localized or clustered markers.

FIG. 18 illustrates one embodiment of a cell imaging application of the disclosure, using interacted markers or protein dimers, and providing real-time monitoring of the dynamic processes on cell surfaces. Shown is an illustration of cell surface receptor dimerization, clustering, co-localization, or interaction with ligands or with other cell surface markers.

FIG. 19 illustrates one embodiment of the disclosure, providing real-time monitoring of the dynamic change of cell surface markers, in response to an inducer.

FIG. 20 illustrates one embodiment of the disclosure, providing real-time monitoring of the dynamic processes on cell surfaces. Shown is an illustration of the induction of a target cell to secrete desired targets, including but not limited to proteins or other molecules.

FIG. 21 illustrates one embodiment of the disclosure, providing a method of probing the dynamics of cell surface molecules using DNA sensors, including the use of a beacon on a cell.

FIG. 22 illustrates one embodiment of the disclosure, providing a method of probing the dynamics of secreted molecules using DNA sensors, including the use of a beacon on a cell.

FIG. 23 illustrates monitoring the toehold-mediated DNA strand displacement. (A) Schematic showing the principle of toehold-mediated DNA strand displacement; (B) Kinetic profiles of toehold-mediated DNA strand displacement obtained from the use of six different target concentrations; (C) Increase of fluorescence intensity as a function of the concentrations of the target DNA TC (T8C20). The reaction mixture contained 20 nM DNA probe T*C*:C, and varying concentrations of the target DNATC.

FIG. 24 illustrates (A) Binding of streptavidin to the two biotin-conjugated DNA probes resulted in the formation of TWJ and the displacement of the fluorescence quencher C. (B) Real-time monitoring of the fluorescence increase due to the binding-induced TWJ. The positive control (P.C.) contained 10 nM probe T8C20 and 20 nMT*C*:C in TE-Mg buffer. The negative control (N.C.) contained only 20 nMT*C*:C in TE-Mg buffer. The tested concentration range of the target streptavidin was from 0.16 nM to 10 nM. (C) Increases in fluorescence signals as a function of the concentrations of streptavidin. Fluorescence was measured after the reaction mixture was incubated at 25° C. for 60 min.

FIG. 25 illustrates the principle of the binding-induced DNA strand displacement strategy. Two DNA motifs (OT and C) are designed to bind to the same target molecule through a specific affinity ligand that is conjugated to the ends of both motifs. The OT motif is formed by prehybridizing the output DNA O with the supporting DNA T. Binding of the two affinity ligands to the same target molecule assembles two DNA motifs together, triggering an internal DNA strand displacement reaction between OT and C. As a result, O is released from T, and a subsequent dynamic DNA assembly can be initiated by the released O.

FIG. 26 illustrates a native PAGE analysis of oligonucleotides from the binding-induced DNA strand displacement. Lane 1, low molecular DNA ladder; lane 2, 2 μM OT; lane 3, 2 μM C; lane 4, from analysis of a mixture containing 2 μM OT and 2 μM C; lane 5, from analysis of a mixture containing 2 μM OT, 2 μM C, and 1 μM streptavidin

FIG. 27. (A) Principle of the binding-induced strand displacement beacon. (B) Evaluation of the binding-induced displacement beacon. The fluorescence intensity was normalized such that 1 normalized unit (n.u.) corresponds to 1 nM O. Control-1 contained the same amount of streptavidin and reagents, except that 500 μM biotin was used to saturate all the binding sites of streptavidin. Control-2 was carried out using the same amount of streptavidin and reagents with the streptavidin sample solution, but without O. Similarly, Control-3 was carried out without competing DNA C, and Control-4 was carried out without OT. In the blank, all reagents were the same as in the streptavidin sample solution, except that there was no streptavidin. Positive control (P.C.), 10 nM 0, 20 nM FQ in TE-Mg buffer; negative control (N.C.), only 20 nM FQ in TE-Mg buffer.

FIG. 28. Estimation of the conversion efficiency from target streptavidin to O at different streptavidin concentrations through the binding-induced displacement beacon. The streptavidin test solutions contained 20 nM OT, 20 nM C, 20 nM QF, and varying concentrations of streptavidin. Error bars represent one standard deviation from duplicate analyses.

FIG. 29 illustrates the optimization of the binding-induced DNA strand displacement to minimize the target-independent strand displacement. Streptavidin test solutions contained 5 nM streptavidin, 10 nM OT, 10 nM C, and 20 nM FQ. In the blank, all reagents were the same as streptavidin sample solution, but with no streptavidin added. Effects of simultaneous increases in the length of both OT and C on the performance of the binding-induced strand displacement were monitored at 45 (A) and 150 min (B). Effects of the length difference between OT and C were also monitored at 45 (C) and 150 min (D). The negative control (N.C.) contained only 20 nM FQ in TE-Mg buffer. Error bars represent one standard deviation from duplicated analyses.

FIG. 30. A) Principle of the binding-induced catalytic DNA circuit. Evaluation of the binding-induced catalytic DNA circuit. The fluorescence intensity was normalized such that 1 n.u. corresponds to 1 nM positive DNA P (details in the SI). An output DNA test solution contained 10 nM output DNA O, 125 nM H1, 200 nM H2, and 125 nM F′Q′. Streptavidin test solutions contained 20 nM OT, 20 nM C, 125 nM H1, 200 nM H2, 125 nM F′Q′, and varying concentrations of streptavidin. In the blank, all reagents were the same as in the streptavidin test solutions, but without streptavidin. (C) Increases in fluorescence intensity reflect increasing concentrations of streptavidin that converts to positive DNA P by the binding-induced catalytic DNA circuit. The magnitude of amplification was determined by the linear fitting between fluorescence intensity and concentration of streptavidin. Error bars represent one standard deviation from duplicated analyses.

FIG. 31 provides a schematic illustrating that binding of streptavidin to biotinylated DNA results in binding-induced DNA assembly and strand displacement of the output DNA O. The supporting DNA T and the competing C were each conjugated with a biotin molecule. T was initially hybridized to the output DNA O, forming the OT motif. Binding of the two biotinylated DNA with the same target streptavidin molecule brought C in close proximity to OT. This process increased the local concentration of C drastically, and thus accelerated the strand displacement between C and OT. As a result, O was released from T as an output to trigger a subsequent DNA assembly.

FIG. 32 shows native PAGE analysis of the binding-induced DNA strand displacement with output DNA L (50 nt in length). Lane 1 contained low molecular DNA ladder. Lane 2 contained 2 μM L. Lane 3 contained 2 μM LT. Lane 4 was from the analysis of a mixture containing 2 μM LT and 2 μM C. Lane 5 was from the analysis of a mixture containing 2 μM LT, 2 μM C, and 1 μM streptavidin.

FIG. 33 shows the characterization of the toehold-mediated strand displacement beacon that was able to response to the output DNA O. The reaction mixture contained 20 nM FQ and varying concentrations of the output DNA O.

FIG. 34 provides a schematic showing the model for calculating the theoretical concentrations of the released output DNA O. As each streptavidin molecule contain 4 binding sites for biotin, a maximum of 2 output DNA molecules can be released from 1 streptavidin molecule. This is achieved by having 2 OT duplexes and 2 C molecules binding to the same streptavidin molecule (e.g. shown in A). When 3 OT duplexes and 1 C molecule bind to the same streptavidin molecule, only 1 output DNA molecule can be released (e.g. shown in B). Similarly, 1 output DNA molecule can be released when 3 C molecules and 1 OT duplex bind to the same streptavidin molecule (e.g. shown in C). When 4 OT duplexes (D) or 4 C molecules (E) bind to the same streptavidin molecule, no output DNA can be released. As each streptavidin have 4 binding sites, so there are 16 possible binding complexes in total. Based on the frequencies of each type of binding structures shown in A to E, the determined possibility for each streptavidin molecule to form one effective binding complex that can result in the release of one output DNA molecule is 1.25.

FIG. 35 shows the optimization on the ratio between T and O. (A) Characterization of OT using PAGE. Lane 1 contained low molecular DNA ladder. Lane 2 contained 2 μM O. Lane 3 contained 2 μM T. Lane 4 contained a mixture of 2 μM T and 2 μM O. Lane 5 contained a mixture of 2 μM T, 1.5 μM O. Lane 6 contained a mixture of 2 μM T and 1.3 μM O. Lane 7 contained a mixture of 2 μM T and 1 μM O. (B) Characterization of OT using binding-induced displacement beacon. The streptavindin test solutions contained 10 nM streptavidin, 10 nM C, 10 nM QF, and OT with varying ratios. Different ratios between T and O were achieved by fixing the concentration of T at 10 nM and tuning the concentrations of O. Error bars represent one standard deviation from duplicated analyses.

FIG. 36. (A) Binding-induced strand-displacement beacon for the detection of PDGF-BB. The DNA probes OT and C were each extended with a PDGF aptamer, forming Apt-OT and Apt-C. The binding of PDGF-BB to its aptamer resulted in binding-induced DNA assembly and strand displacement of the output DNA O. The output DNA O released from Apt-T triggered a subsequent DNA strand displacement to release F from FQ, and thus turned on the fluorescence. (B) Increases in fluorescence signal as a function of concentrations of PDGF-BB.

FIG. 37 shows native PAGE analysis of the binding-induced DNA strand displacement showing the elimination of the target-independent displacement. The OT probe used in this experiment was 14 by in length, and competing DNA C was 12 nt in length. Lane 1 contained low molecular DNA ladder. Lane 2 contained 2 μM OT. Lane 3 contained 2 μM C. Lane 4 was from the analysis of a mixture containing 2 μM OT and 2 μM C. Lane 5 was from the analysis of a mixture containing 2 μM OT, 2 μM C, and 1 μM streptavidin.

FIG. 38. Characterization of toehold-mediated catalytic DNA circuit that can respond to the output DNA O. The O test solution contained 10 nM 0, 125 nM H1, 200 nM H2, and 125 nM F′Q′. A positive control contained 50 nM positive DNA P, 125 nM H1, 200 nM H2, and 125 nM F′Q′. A negative control contained the same reagents of O test solution, except there was no O added.

FIG. 39 illustrates one embodiment of the disclosure, providing real-time imaging of a cell surface marker.

FIG. 40 shows cells directly observed under confocal fluorescence microscope. FAM-labeled sensors are visible for both sample (top row) and isotype control (bottom row). The isotype control was prepared and treated using the same condition as for the sample, except that non-specific goat IgG was used to modify DNA probes instead of HER2-specific goat IgG.

### DETAILED DESCRIPTION

As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, or characteristic, but not every embodiment necessarily includes that aspect, feature, structure, or characteristic. Moreover, such phrases may, but do not necessarily, refer to the same embodiment referred to in other portions of the specification. Further, when a particular aspect, feature, structure, or characteristic is described in connection with an embodiment, it is within the knowledge of one skilled in the art to affect or connect such aspect, feature, structure, or characteristic with other embodiments, whether or not explicitly described.

The singular forms “a,” “an,” and “the” include plural reference unless the context clearly dictates otherwise. Thus, for example, a reference to “a plant” includes a plurality of such plants. It is further noted that the claims may be drafted to exclude any optional element. As such, this statement is intended to serve as antecedent basis for the use of exclusive terminology, such as “solely,” “only,” and the like, in connection with the recitation of claim elements or use of a “negative” limitation.

The term “and/or” means any one of the items, any combination of the items, or all of the items with which this term is associated. The phrase “one or more” is readily understood by one of skill in the art, particularly when read in context of its usage.

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values and ranges proximate to the recited range that are equivalent in terms of the functionality of the composition, or the embodiment.

As will be understood by the skilled artisan, all numbers, including those expressing quantities of reagents or ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percents or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc.

As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, as used in an explicit negative limitation.

The term “contacting” refers to the act of touching, making contact, or of bringing to immediate or close proximity, including at the cellular or molecular level, for example, to bring about a physiological reaction, a chemical reaction, or a physical change, in vitro, or in vivo.

An “effective amount” refers to an amount effective to bring about a recited effect.

As used herein, the term “nucleic acid” and “polynucleotide” refers to deoxyribonucleotides or ribonucleotides and polymers thereof in either single- or double-stranded form, composed of monomers (nucleotides) containing a sugar, phosphate and a base that is either a purine or pyrimidine. Unless specifically limited, the term encompasses nucleic acids containing known analogs of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucl. Acids Res., 19:508 (1991); Ohtsuka et al., J. Biol. Chem., 260:2605 (1985); Rossolini et al., Mol. Cell, Probes, 8:91 (1994).

Deoxyribonucleic acid (DNA) in the majority of organisms is the genetic material while ribonucleic acid (RNA) is involved in the transfer of information contained within DNA into proteins. The term “nucleotide sequence” refers to a polymer of DNA or RNA that can be single- or double-stranded, optionally containing synthetic, non-natural or altered nucleotide bases capable of incorporation into DNA or RNA polymers. A “nucleic acid fragment” is a fraction or a portion of a given nucleic acid molecule.

The terms “nucleic acid”, “nucleic acid molecule”, “nucleic acid fragment”, “nucleic acid sequence or segment”, or “polynucleotide” may also be used interchangeably with gene, cDNA, DNA and RNA encoded by a gene, e.g., genomic DNA, and even synthetic DNA sequences. The term also includes sequences that include any of the known base analogs of DNA and RNA.

The term “gene” is used broadly to refer to any segment of nucleic acid associated with a biological function. Thus, genes include coding sequences and/or the regulatory sequences required for their expression. For example, gene refers to a nucleic acid fragment that expresses mRNA, functional RNA, or specific protein, including regulatory sequences. Genes also include nonexpressed DNA segments that, for example, form recognition sequences for other proteins. Genes can be obtained from a variety of sources, including cloning from a source of interest or synthesizing from known or predicted sequence information, and may include sequences designed to have desired parameters.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis that encode the native protein, as well as those that encode a polypeptide having amino acid substitutions.

“Operably-linked” refers to the association of nucleic acid sequences on single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation.

As used herein, “TWJ” refers to DNA three way junctions.

As used herein, “in situ” refers to in the natural or normal place, confined to the site of origin without invasion of neighboring tissues, or in the original or natural place or site.

As used herein, “antigen” refers to any substance, including proteins, that when recognized as non-self or foreign by the adaptive immune system triggers an immune response, stimulating the production of an antibody that specifically reacts with it.

As used herein, “biological sample” refers to any sample derived from a human, animal, plant, bacteria, fungus, virus, or yeast cell, including but not limited to tissue, blood, bodily fluids, serum, sputum, mucus, bone marrow, stem cells, lymph fluid, secretions, and the like.

As used herein, “biological material” refers to the object to be sensed or detected by the techniques, methods, systems, and technologies provided herein. Biological material, thus, can be proteins, DNA, RNA, any genetic material, small molecules, or any moiety to be detected by the techniques, methods, systems, and technologies provided herein.

As used herein, “trace levels” refer to very small quantities of a substance or material.

As used herein, “point-of-care” diagnostics or tests refer to analytical methods and tests that can be performed near the patient, including but not limited to, at the clinic, at the bedside, in the operating room, in the procedure room, in the laboratory or any other test that can be done in a near location to the patient or subject of interest.

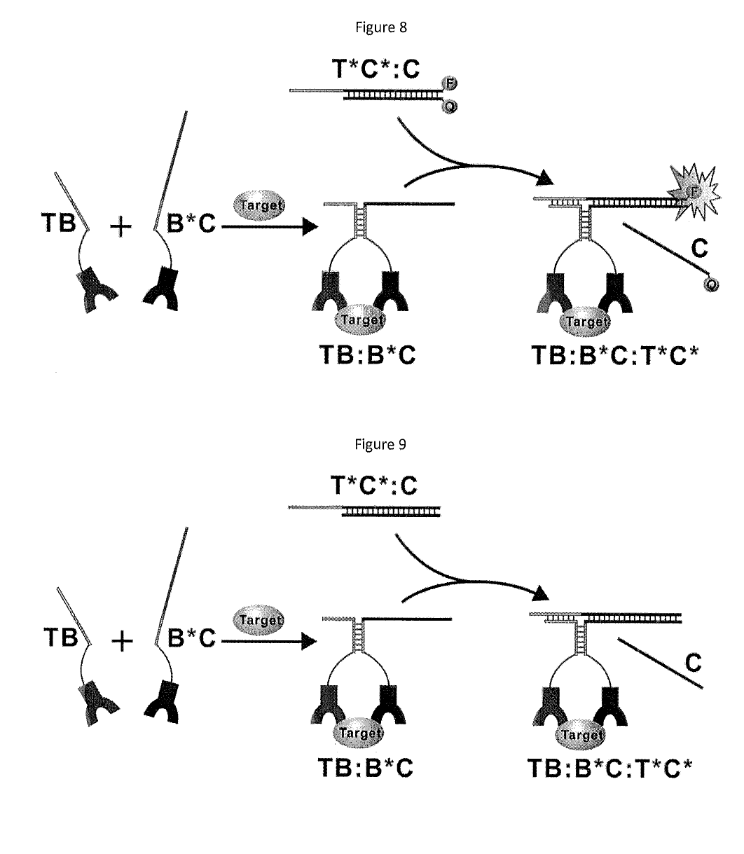

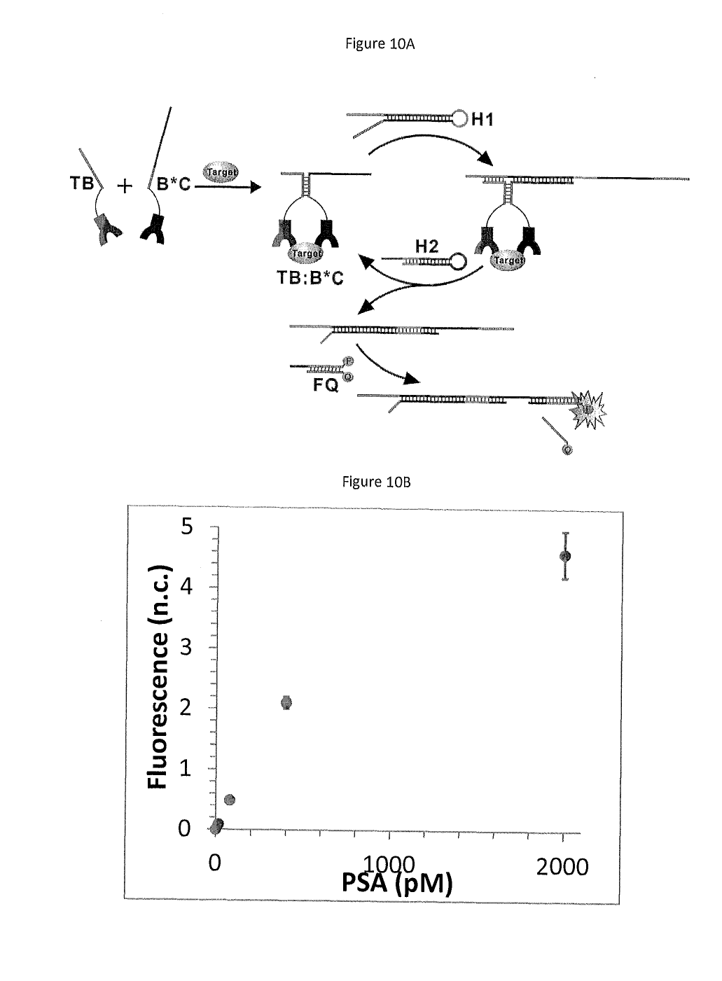

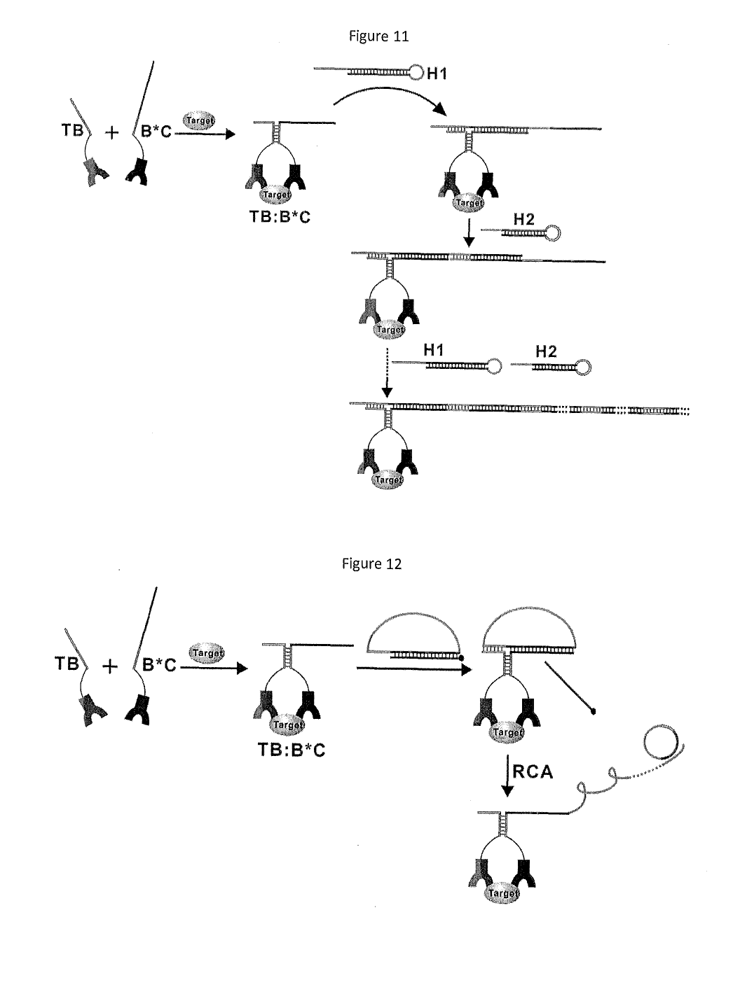

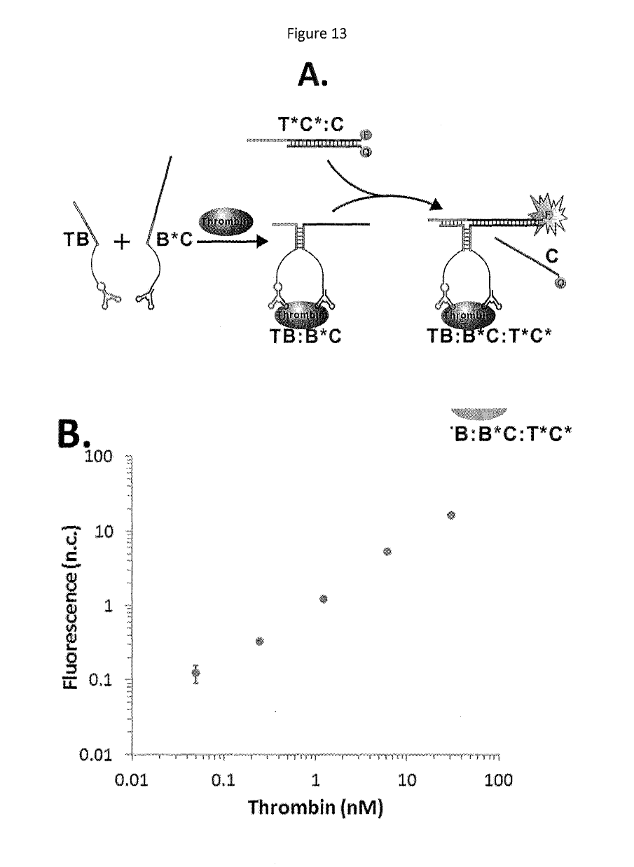

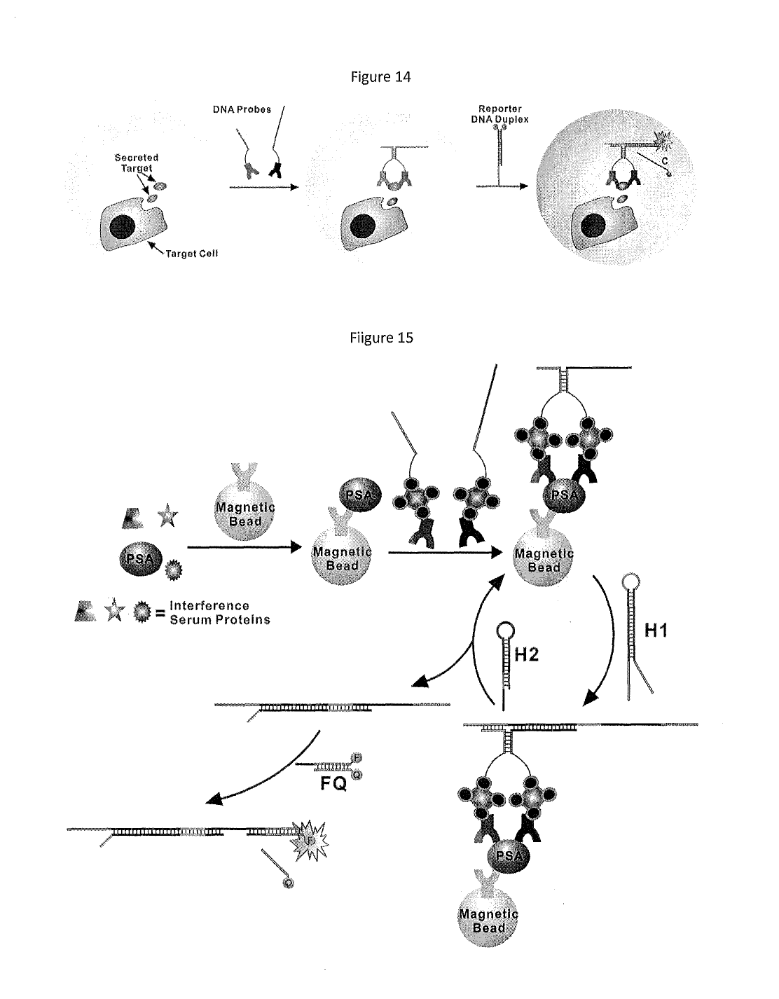

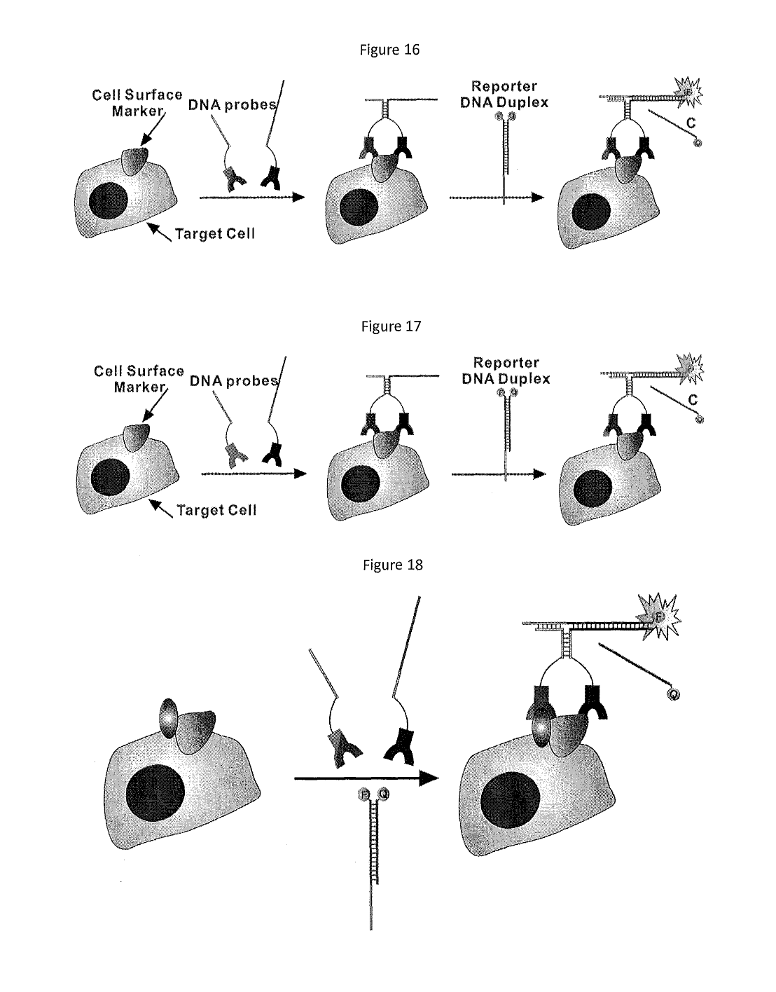

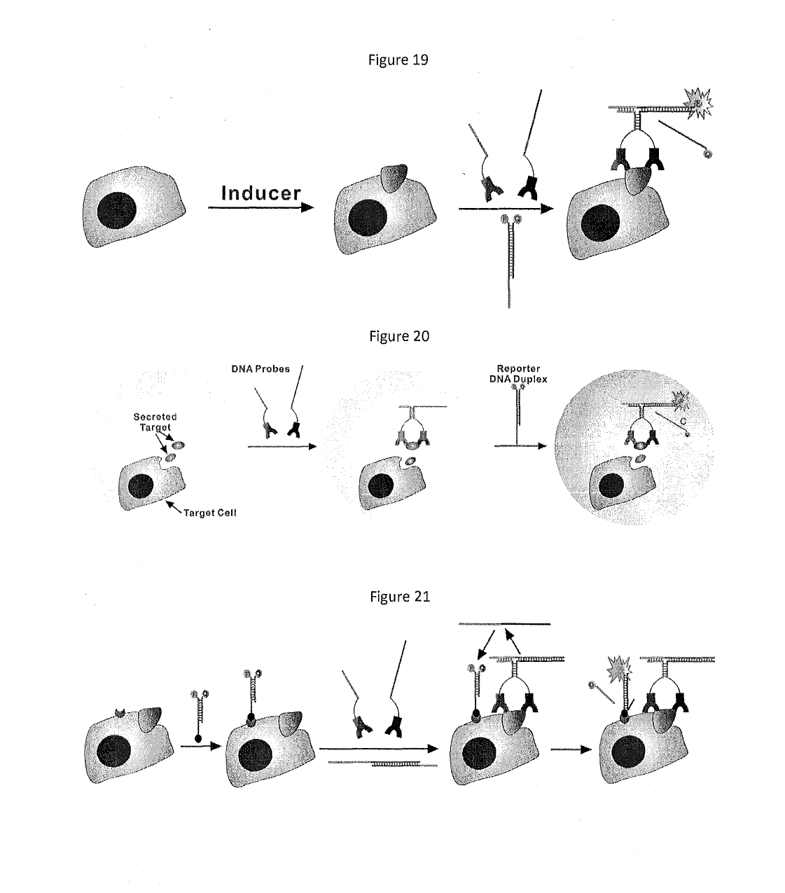

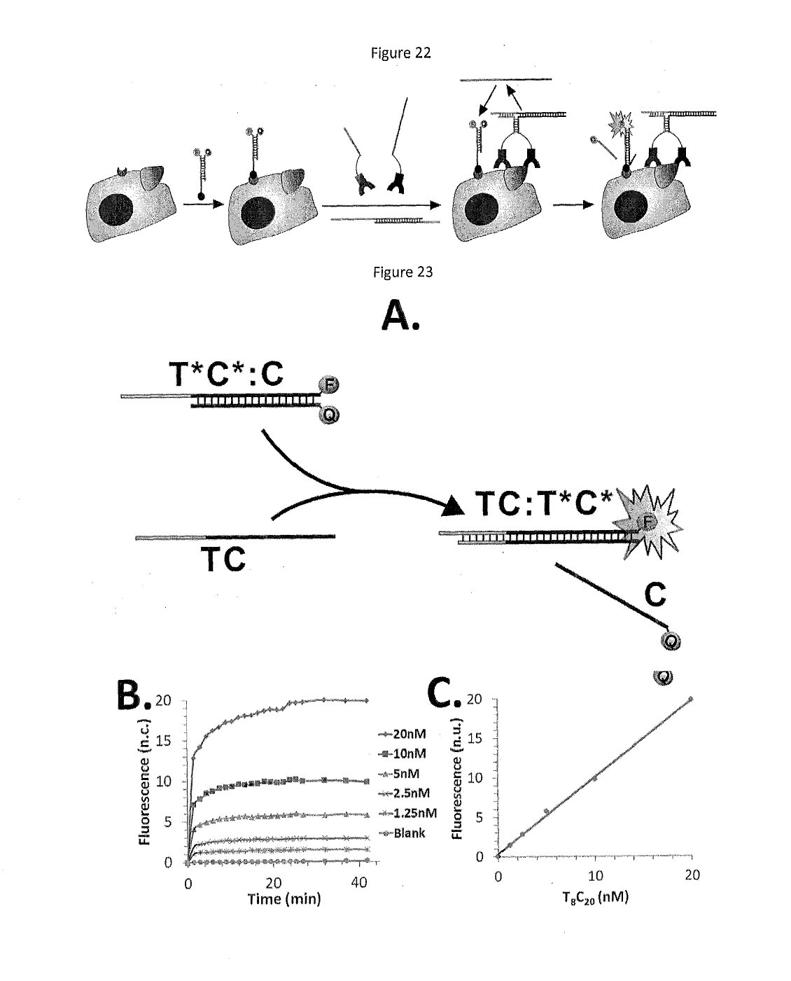

Embodiments of the present invention are based on the observation that the affinity bindings between target molecules and their ligands could serve as a trigger to the formation of DNA TWJs. This binding-induced TWJ may provide a strategy to design protein-responsive DNA devices and assemblies. This binding-induced TWJ technology has origins in the knowledge that two separate DNA strands that are linked by a stable DNA duplex can facilitate toehold-mediated DNA strand displacements (associative DNA toehold). This TWJ strategy is highly successful for DNA, but its application to proteins was not available and was challenging. Applicants' innovation to confront this challenge led to the development of a binding-induced TWJ technique, one example of which is illustrated schematically in FIG. 8. FIG. 8 shows two DNA motifs, TB and B*C, and each is conjugated with an affinity ligand. Motif TB is designed to have a toehold domain T and a binding domain B, separated by a flexible linker of two thymidine bases. Motif B*C has a binding domain B* and a competing domain C. In one embodiment, TB and B*C are designed to have only 6 complementary bases (domain B and B*, green color in FIG. 9), so that they cannot form a stable duplex at room temperature. However, in the presence of the target molecule, the binding of two affinity ligands to the same target molecule brings TB and B*C to close proximity, greatly increasing their local effective concentrations. Consequently, TB and B*C hybridize to each other to form a stable TB:B*C duplex. Once TB:B*C forms, it triggers a subsequent toehold-mediated strand displacement reaction with detection probe T*C*:C, forming a binding-induced TWJ (TB:B*C:T*C*) and displacing the motif C.

Thus, the binding-induced DNA TWJ strategy is able to convert protein bindings to the formation of DNA TWJ. The binding-induced DNA TWJ makes use of two DNA motifs each conjugated to an affinity ligand. The binding of two affinity ligands to the target molecule triggers assembly of the DNA motifs and initiates the subsequent DNA strand displacement, resulting in a binding-induced TWJ. In one embodiment, real-time fluorescence monitoring of the binding-induced TWJ enables highly sensitive detection of the specific protein targets. For example, a detection limit of 2.8 ng/mL was achieved for prostate specific antigen (PSA).

The binding-induced TWJ approach compares favorably with the toehold-mediated DNA strand-displacement, the associative (combinative) toehold-mediated DNA strand-displacement, and the binding-induced DNA strand-displacement. The binding-induced TWJ may broaden the scope of dynamic DNA assemblies and provide a new strategy to design protein-responsive DNA devices and assemblies.

The ability to generate detection signals with high sensitivity and fast kinetics in homogeneous solutions without the need for enzymes or thermal cycling makes these methods suitable for many emerging applications, including but not limited to, point-of-care disease diagnostics and molecular imaging in live cells. This novel approach to accelerate DNA strand displacement reactions through affinity binding to specific proteins may open up opportunities to further expand the state-of-art DNA nanotechnology to proteins for diverse applications.

In one embodiment, it is preferred to have the DNA strand displacement process faster than the dissociation of the target from affinity ligands. This can be achieved by using affinity ligands with slow dissociation rates, e.g., slow off-rate modified aptamer (SOMAmer); stabilizing the binding complex by photo or chemical cross-linking; and/or increasing the rate of intramolecular DNA strand displacement by tuning the length of DNA probes or increasing the incubation temperature.

In one embodiment, the invention uses sandwiched binding among one target and two binders to form a DNA-ligand-target complex. This complex then triggers a DNA strand displacement reaction. The target may be any protein, protein complex, protein-protein interaction, protein-DNA interaction, cancer cell, stem cell, blood cell, bacteria cell, fungal cell, yeast cell, animal cell, plant cell, virus, virus particle, or any small molecule of interest. The targets may be present in buffer, cell culture media, human or animal biopsy, including but not limited to, blood, serum, plasma, serum, bone marrow, urine, sputum, saliva, tears, mucus, or any bodily fluid or tissue.

Suitable affinity ligands may include, but are not limited to, antibodies, small molecules, lectin, aptamers, or any molecule that can bind to the target. In one embodiment, the affinity ligand comprises polyclonal antibodies to the target. Because the binding between the first DNA motif and the second DNA motif is brought about by binding to the target in close proximity, it is not suitable for the first and second affinity ligands to be specific to the same epitope. Thus, if monoclonal antibodies are used, then they are preferably directed to different epitopes on the same target.

The displacement probe DNA (T*C*:C) may be linear, hairpin or circular DNA, as well as any fragment of DNA. These methods may offer in situ signal generation, in situ signal amplification, fully tunable kinetics, versatility to a wide range of target-binder pairs, isothermal versatility (can be performed at room temperature), flexible target amplification strategies, and other features as described herein. The kinetics can be tuned from either toehold part or binding part. In certain embodiments, by tuning the binding part, a signal can be generated instantly upon binding to the target.

In one embodiment, the detection probe comprises linear DNA and a displacement beacon. In this embodiment, two strands of DNA are conjugated with a fluorescence donor and a fluorescence acceptor. The donor may be any organic fluorescence molecule, as well as a quantum dot or other suitable donor. The acceptor can be any organic fluorescence molecule, as well as a molecular quencher or a gold nanoparticle, or any other suitable acceptor.

In certain embodiments, the claimed methods can be used as homogenous target detection methods, and applied to diagnostics for diseases or disorders, real-time sensing for specific biological processes, detection of certain targets (including, but not limited to, toxins, pathogens, and the like) in environmental, food, or other biological samples. In some embodiments, Applicants' methods can be used to detect human alpha-thrombin. In other embodiments, Applicants' methods can be used to detect PSA (prostate specific antigen) in biological samples, such as serum samples or other samples.

In other embodiments, the claimed methods can be used as real-time detection methods for secreted targets from target cells. In this embodiment, the methods can be used to sense or detect certain biological processes in a biological sample. The target cell can be from cell culture, tissue samples, in vivo samples, in microfluidic chambers, and an emulsion droplet, as well as from other origins of target cells.

In certain embodiments, claimed methods can be used as heterogenous target detection methods. Targets to be detected may be present on solid supports, including but not limited to beads, glass slides, arrays, chips, tissue samples, poly or composite slides, or other solid supports. In a specific embodiment, Applicants' methods can be used to detect PSA on magnetic beads, as well as glass slides, arrays, chips, tissue samples, cell surfaces, poly or composite slides, or other solid supports.

In other embodiments, the claimed methods can be used for cell imaging applications. In certain embodiments, these methods can be used to detect a cell surface marker on a target cell. The target cell can be any cell of interest, including but not limited to cancer cells, stem cells, bacteria cells, fungal cells, blood cells, or any bodily cells, animal cells, yeast cells or plant cells. The cell surface markers can be proteins, carbohydrates, protein dimers, protein complexes, protein clusters, protein-protein interactions, antigens, and any other cell surface markers. In other cell imaging applications, the methods as provided herein can detect co-localized and/or clustered markers. In these methods, the cells can be any cells of interest, including but not limited to cancer cells, stem cells, bacteria cells, fungal cells, blood cells, or any bodily cells, animal cells, yeast cells or plant cells. In still other embodiments, the cell imaging methods can be used to detect interacted markers on cell surfaces and/or protein dimers on cell surfaces. In these methods, the cells can be any cells of interest, including but not limited to cancer cells, stem cells, bacteria cells, fungal cells, blood cells, or any bodily cells, animal cells, yeast cells or plant cells.

In one embodiment, using a beacon in the solution, the biology of cell surface and cell-secreted molecules can be analysed using DNA sensors. The claimed methods may allow real-time monitoring of the dynamic processes on cellular surfaces, including real-time cell surface marker monitoring, and real-time monitoring of cell surface receptor dimerization, clustering, co-localization or interaction with ligands or other cell surface markers. The claimed methods may allow the ability to induce a cell to secrete proteins or other molecules or substances, in real time.

In one embodiment, using a beacon on the cell itself, the biology of cell surface and cell-secreted molecules can be analysed using DNA sensors, which may enable decision making sensing based on cell surface markers. As a non-limiting example, the methods described herein may be able to sense or detect when two markers turn on one beacon, revealing a negative marker inhibiting the turning on of a beacon. The methods described herein may also enable decision making sorting of cells. As a non-limiting example, magnetic beads, microfluidic chips, or RCA chips can be conjugated with DNA probes to sort cells with desired marker combinations. Applicants' methods and techniques also enable in vivo sensing for epithelial cells or solid tumors. By using Applicants' base technique with a beacon on the cell itself, one can trace the origin and destiny of the secreted proteins by turning on the surrounding cells.

The methods described herein may provide for the amplification and detection or sensing of proteins and other molecules with extremely high sensitivity. In one embodiment, a novel sensor is provided which enables sensitive and real-time detection of specific target molecules and cells in situ. The methods described herein are applicable to many fields of study, as they are not temperature dependent, and provide enzyme-free signal generation and amplification ability in homogeneous and heterogenous solutions or samples.

The methods described herein may provide advantages over existing methods. For example, these methods provide for signal amplification at room temperature, thus the need for thermal cycling is absent, or provide for signal amplification without the use of enzymes. These advantages may enable point-of-care diagnostic applications. In addition, these methods do not require separation of the sample or washing steps, and the techniques and methods can be performed in situ. These methods can be performed in situ with no need for separation, making these tools desirable for imaging applications. Because these methods can be performed in situ, they are well suited for imaging applications, such as live cell imaging or tissue staining. In imaging applications, for example, these methods provide the ability to perform live cell imaging or tissue staining. This is in contrast to current methods and techniques, such as proximity ligation assays. Current methods rely strongly on enzyme driven reactions (such as the polymerase chain reaction and rolling circle amplification), and thus are not suitable for point-of-care diagnostic applications or live cell imaging applications.

In certain aspects, the invention may comprise reagents, reagent kits, probes, imaging probes and diagnostic assays. Assays utilizing these reagents, reagent kits, or probes, can detect trace levels of target protein markers and target cells. The reagent kits can be used as probes for imaging, for detecting specific proteins or protein-protein interaction in live cells or tissues. In one embodiment, the claimed methods and techniques provide real-time detection of subnanomolar amounts of a target, as demonstrated by the detection of streptavidin and PSA as described below. The reagent kits can be formulated for detection of any desired protein or marker.

In other embodiments, signalling aptamer sensors can also provide real-time detection probes for target molecules in patient samples or in live cells. In the claimed techniques and methods, aptamers, antibodies and other probes can be used.

In certain embodiments, the methods described herein can be used for diagnostic purposes and applications, including point of care diagnostic applications.

### EXAMPLES

The following examples are intended only to illustrate specific embodiments of the invention.

Materials and Reagents

Streptavidin from Streptomyces avidinii (product number, S4762), biotin (product number, B4501), bovine serum albumin (BSA), prostate specific antigen from human semen (PSA), sterile-filtered human serum, magnesium chloride hexahydrate (MgCl2.6H2O), and 100× Tris-EDTA (TE, pH 7.4) buffer were purchased from Sigma (Oakville, ON, Canada). SYBR Gold and ROX Reference Dye (ROX) were purchased from Life Technologies (Carlsbad, Calif.). Biotinylated Human Kallikrein 3/PSA polyclonal antibody (goat IgG) was purchased from R&D systems (Burlington, ON, Canada). Reagents for polyacrylamide gel electrophoresis (PAGE), including 40% acrylamide mix solution and ammonium persulfate were purchased from BioRad Laboratories (Mississauga, ON, Canada). Tween 20 and 1, 2-bis(dimethylamino)-ethane (TEMED) were purchased from Fisher Scientific (Nepean, ON, Canada). NANOpure H₂O (>18.0 M), purified using an Ultrapure Milli-Q water system, was used for all experiments. All DNA samples were purchased from Integrated DNA Technologies (Coralville, Iowa) and purified by HPLC. The DNA sequences and modifications are listed in Tables 1 and 2.

TABLE 1
DNA sequences and modifications for constructing binding-induced
DNA three-way junctions. Toehold domains are underlined. C domains
are in bold. Binding domains are italicized.

| DNA Name | | Sequences |
|---|---|---|
| T*C* | | 5′-CTA GAG CAT CAC ACG GAC ACA TGG GAT ACA CGC TT-FAM-3′ |
| | | [SEQ ID NO: 1] |
| C | | 5′-Dabcyl-AA GCG TGT ATC CCA TGT GTC-3′ |
| | | [SEQ ID NO: 2] |
| B*C | | 5′-AA GCG TGT ATC CCA TGT GTC-CCT CAC TGA GAC TCC-TTT TTT T-Biotin-3′ |
| | | [SEQ ID NO: 3] |
| TB | T₁₅B₆ | 5′-Biotin-TTT TTT TTTTTTTTT T-GTG AGG-TT-CGT GTG ATG CTC TAG-3′ |
| | | [SEQ ID NO: 4] |
| | T₉B₆ | 5′-Biotin-TTT TTT TTTTTTTTT-GTG AGG-TT-CGT GTG ATG-3′ |
| | | [SEQ ID NO: 5] |
| | T₈B₆ | 5′-Biotin-TTT TTT TTTTTTTTT T-GTG AGG-TT-CGT GTG AT-3′ |
| | | [SEQ ID NO: 6] |
| | T₇B₆ | 5′-Biotin-TTT TTT TTTTTTTTT T-GTG AGG-TT-CGT GTG A-3′ |
| | | [SEQ ID NO: 7] |
| | T₆B₆ | 5′-Biotin-TTT TTT TTTTTTTTT T-GTG AGG-TT-CGT GTG-3′ |
| | | [SEQ ID NO: 8] |
| T₉B₁₅ | | 5′-GGA GTC TCA GTG AGG-TT-CGT GTG ATG-3′ |
| | | [SEQ ID NO: 9] |
| TC(T₈C₂₀) | | 5′-AA GCG TGT ATC CCA TGT GTCCGTGTGAT-3′ |
| | | [SEQ ID NO: 10] |
| Biotin-C* | | 5′-Biotin-TTT TTT TTTTTTTTTGAC ACA TGG GAT ACA CGC TT-FAM-3′ |
| | | [SEQ ID NO: 11] |
| C-Biotin | | 5′-AA GCG TGT ATC CCA TGT GTC TTT TTTTTTTTT TTT-Biotin-3′ |
| | | [SEQ ID NO: 12] |

Probe Preparation for Binding-Induced DNA Three-Way Junction

DNA probe (T*C*:C) for binding-induced TWJ (Table 1) was prepared at a final concentration of 5 pM by mixing 20 μL 50 μM 6-carboxyfluorescein-labeled (FAM) T*C* with 20 μL 100 μM dark quencher-labeled C in 160 μL TE-Mg buffer (1×TE, 10 mM MgCl₂, 0.05% Tween20). The mixture was heated to 90° C. for 5 min and then the solution was allowed to cool down slowly to 25° C. in a period of 3 hours. Probe (T*C*:C) for gel electrophoresis (Table S2) was also prepared at a final concentration of 5 μM by mixing 20 μL 50 μM unlabeled T*C* with 20 μL 25 μM FAM-labelled C in 160 μL TE-Mg buffer. Similarly, the solution was heated to 90° C. for 5 min, and then cooled down to 25° C. slowly in a period of 3 hours.

Real-Time Monitoring of the Toehold-Mediated DNA Strand Displacement

For a typical toehold-mediated DNA strand displacement reaction (FIG. 23), the reaction mixture contained 20 nM probe T*C*:C, 50 nM ROX reference dye, 1 μM polyT oligo, varying concentrations of the target DNA TC (T₈C₂₀), and TE-Mg buffer. The reaction mixture was incubated at 25° C. for 45 min in a 96-well plate. Fluorescence was measured directly from the microplate using a multi-mode microplate reader (DX880, Beckman Coulter). The excitation/emission for the DNA probes were 485/515 nm and the excitation/emission for the ROX reference dye were 535/595 nm.

To monitor the kinetic process of toehold-mediated DNA strand displacement reaction (FIG. 23B), the fluorescence of the reaction mixture was measured every 1.5 min for the first 30 minutes and then every 5 minutes for another 15 minutes. FIG. 23B shows increases in the fluorescence signals over a period of 45 min from the toehold-mediated DNA strand displacement between 20 nM T*C*:C and TC (0-20 nM). A calibration between the fluorescence intensity and the concentration of TC is linear with the range of concentrations tested (1.25-20 nM, FIG. 23C).

Binding-Induced TWJ Probes for Prostate Specific Antigen (PSA) and Human α-Thrombin

To prepare DNA probes for the detection of PSA using binding-induced TWJ, 25 μL 2.5 μM biotinylated probe T₉B₆ or probe B*C was mixed with equal volume of 2.5 μM streptavidin (diluted in 20 mM Tris buffer, containing 0.01% BSA), and then incubated the solution at 37° C. for 30 min, followed by incubation at 25° C. for another 30 min. To this reaction mixture, 50 μL 1.25 μM biotinylated PSA polyclonal antibodies (diluted in 20 mM Tris buffer saline, containing 0.01% BSA) was then added. The solution was incubated at 25° C. for 30 min. The prepared DNA probe was then diluted to 250 nM with a solution containing 20 mM Tris buffer saline, 0.01% BSA, and 1 mM biotin.

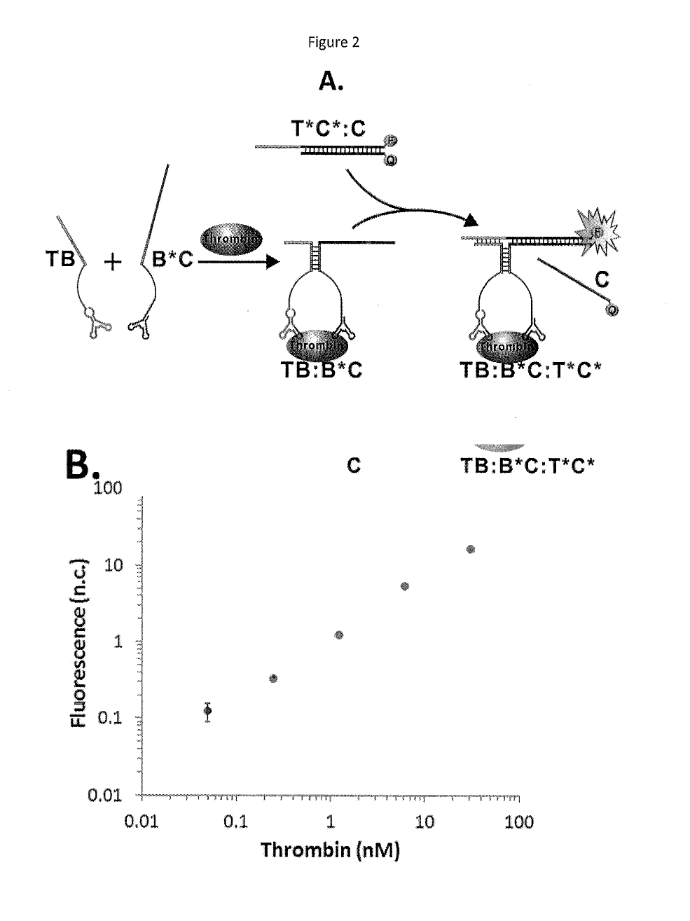

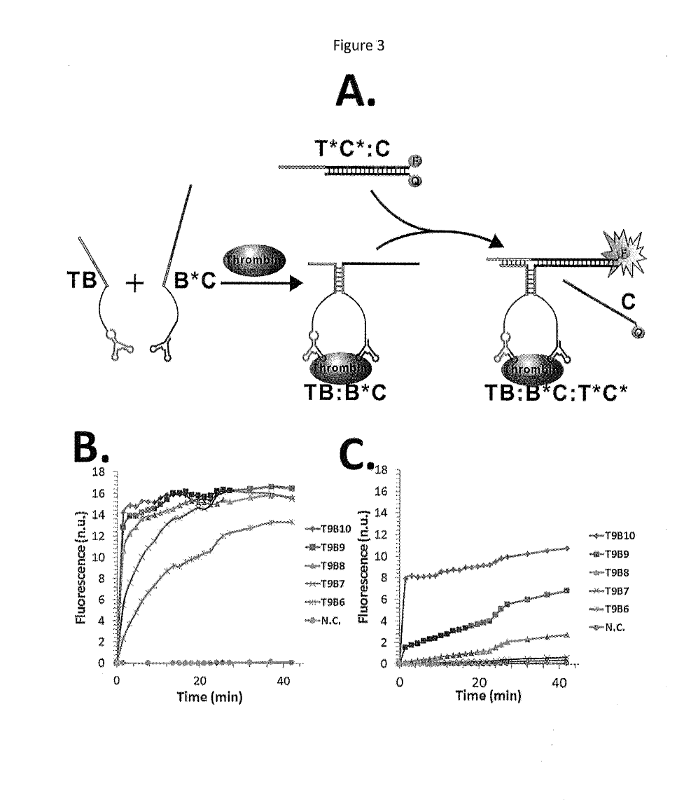

To prepare DNA probes for the detection of thrombin, two distinct thrombin aptamers were directly incorporated to the end of T₉B₆ and B*C during DNA synthesis (FIGS. 2, 3). Detailed DNA sequences and modifications were shown in Table 2.

TABLE 2
DNA sequences and modifications used in thrombin detection.
Aptamer sequences are underlined. C domains are in
bold. Binding domains are italicized.

| DNA name | Sequences |
|---|---|
| B*C | 5′-AA GCG TGT ATC CCA TGT GTC-CCT CAC TGA G-TT TTTTTT TT- |
| | GGT TGG TGT GGT TGG-3′ |
| | [SEQ ID NO: 13] |
| TB | 5′-AGT CCG TGG TAG GGC AGG TTG GGG TGA CT T TTT TTTTTTTTT |
| | T GTG AGG TT CGT GTG ATG-3′ |
| | [SEQ ID NO: 14] |

Detection of PSA and Thrombin Using Binding-Induced TWJ

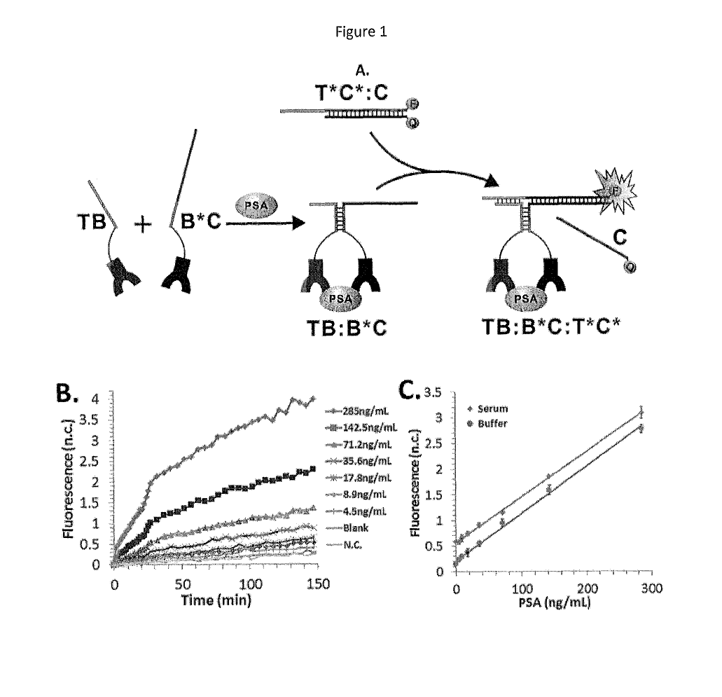

For the detection of PSA or thrombin in buffer or in diluted human serum (FIGS. 1, 2, and 3), the reaction mixture contained 20 nM antibody or aptamer-modified probe TB, 20 nM antibody or aptamer-modified probe B*C, 50 nM ROX reference dye, 1 μM polyT oligo, varying concentrations of the target protein, and TE-Mg buffer. The reaction mixture was incubated at 37° C. for 30 min and then transferred into a 96-well plate, Detection probe T*C*:C was then added to the reaction mixture at a final concentration of 20 nM. Fluorescence was measured every 1.5 min for the first 30 min and then every 5 min for another 2 hours. Fluorescence was measured directly from the microplate using a multi-mode microplate reader (DX880, Beckman Coulter). The excitation/emission for DNA strand displacement were 485/515 and excitation/emission for ROX reference dye were 535/595 nm. The measured fluorescent signal was normalized so that 1 normalized unit (n.u.) of fluorescence corresponded to fluorescent signal generated by 1 nM TC. This normalization was achieved using a positive control containing 10 nM TC, 20 nM T*C*:C, 1 μM polyT oligo, and 50 nM Rox in TE-Mg buffer, and a negative control containing identical reagents in positive control except that there was no TC added.

The end-point detection of target protein was achieved by incubating the reaction mixture at 25° C. for 60 min in a PCR tube in the dark. The reaction mixture was then transferred into a 96-microplate and fluorescence was measured using the multimode microplate reader as described above.

Monitoring the Formation of Binding-Induced TWJ Using Gel Electrophoresis

A reaction mixture contained 2 μM probe B*C, 2 μM probe TB, 1 μM T*C*:C, 1 μM T*C*, 1 μM target protein, and TE-Mg buffer. The reaction mixture was incubated at 25° C. for 30 min. After incubation, the reaction mixture was then assessed using 12% native polyacrylamide gel electrophoresis (PAGE). All the gels were freshly prepared in house. Before loading, DNA samples were mixed with DNA loading buffer on a volume ratio of 5:1. A potential of 12 V/cm was applied for gel electrophoresis separation. After separation, PAGE gels containing DNA were first directly imaged by an ImageQuant 350 (IQ 350) digital imaging system to measure the DNA bands that contain fluorophore labeled DNA, and the same gel was then stained using SYBR Gold and imaged again by the IQ 350 imaging system.

TABLE 3
DNA sequences and modifications used in
the gel electrophoresis experiments.

| DNA name | Sequences |
|---|---|
| T*C* | 5′-CTA GAG CAT CAC ACG GAC ACA TGG GAT |
| | ACA CGC TT-3′ |
| | [SEQ ID NO: 15] |
| C | 5′-FAM-AA GCG TGT ATC CCA TGT GTC-3′ |
| | [SEQ ID NO: 16] |

Real-Time Detection of Streptavidin Using Binding-Induced TWJ

For real-time detection of streptavidin using binding-induced TWJ, the reaction mixture contained 20 nM FAM-labeled probe T*C*:C, 20 nM probe TB, 20 nM probe B*C, 50 nM ROX reference dye, 1 μM polyT oligo, varying concentrations of the target streptavidin, and TE-Mg buffer. The reaction mixture was incubated at 25° C. in a 96-well microplate. Fluorescence was measured directly from the microplate every 1.5 min for the first 30 min and then every 5 min for another 2 hours. The measured fluorescent signal was normalized so that 1 normalized unit (n.u.) of fluorescence corresponded to fluorescent signal generated by 1 nM TC. This normalization was achieved using a positive control containing 10 nM TC, 20 nM T*C*:C, 1 μM polyT oligo, and 50 nM Rox reference dye in TE-Mg buffer, and a negative control containing identical reagents as in positive control except that there was no TC added. The rate constant k_{obs} was determined from the following equation: ln(1−[output]/[input])=k_{obs}×t, where [output] is the normalized fluorescence at each time point, and [input] is the total normalized fluorescence corresponding to the concentrations of target added.

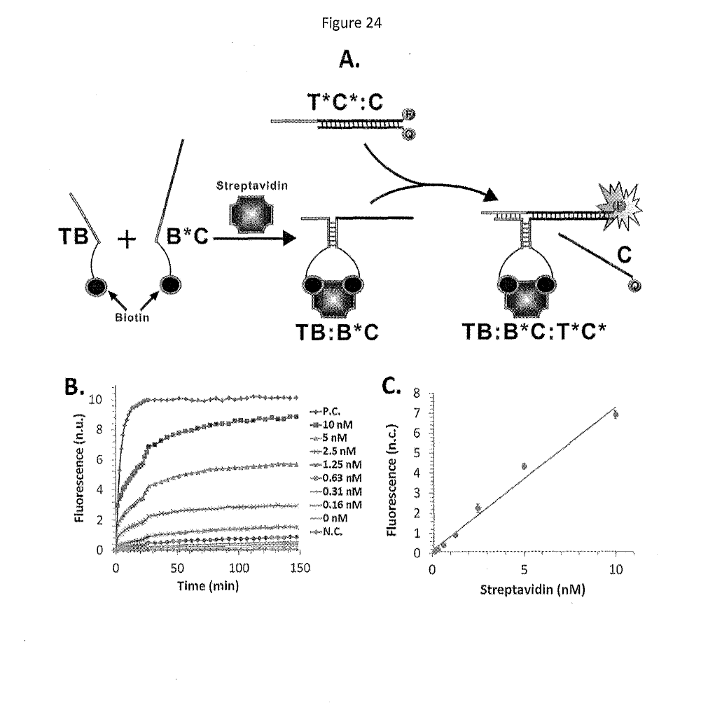

A calibration was generated from the analyses of solutions containing varying concentrations of streptavidin (FIG. 24). The reaction mixtures as described above were incubated in separate PCR tubes in the dark. The reaction mixture was then transferred into a 96-well microplate. Fluorescence was measured as described above.

Monitor the Kinetics of DNA Strand Displacement Mediated by Associative DNA Toehold

For monitoring the kinetics of DNA strand displacement mediated by associative DNA toehold, the reaction mixture contained 20 nM FAM-labeled probe T*C*:C, 10 nM probe T₉B₁₅, 10 nM probe B*C, 50 nM ROX reference dye, 1 μM polyT oligo, and TE-Mg buffer. The reaction mixture was incubated at 25° C. in a 96-well plate. Fluorescence was measured every 1.5 min for the first 30 min and then every 5 min for another 2 hours. The measured fluorescent signal was normalized so that 1 n.u. of fluorescence corresponded to fluorescent signal generated by 1 nM TC. This normalization was achieved using a positive control containing 10 nM T₈C₁₅, 20 nM T*C*:C, 1 μM polyT oligo, and 50 nM Rox in TE-Mg buffer, and a negative control containing identical reagents in positive control except that there was no T₈C₁₅ added. The observed rate constant k_{obs} was determined as described above using equation: ln(1-[output]/[input])=k_{obs}×t.

Monitor the Kinetics of Binding-Induced DNA Strand Displacement

For monitoring the kinetics of binding-induced DNA strand displacement, the reaction mixture contained 20 nM probe Biotin-C*:C, 20 nM probe C-Biotin, 10 nM target streptavidin, 50 nM ROX reference dye, 1 μM polyT oligo, and TE-Mg buffer. The reaction mixture was incubated at 25° C. in a 96-well plate. Fluorescence was measured every 1.5 min for the first 30 min and then every 5 min for another 2 hours. The measured fluorescent signal was normalized so that 1 n.u. of fluorescence corresponded to fluorescent signal generated by 1 nM TC. This normalization was achieved using a positive control containing 10 nM T₈C₁₅, 20 nM T*C*:C, 1 μM polyT oligo, and 50 nM Rox in TE-Mg buffer, and a negative control containing identical reagents in positive control except that there was no T₈C₁₅ added. The observed rate constant k_{obs} was determined as described above using equation: ln(1−[output]/[input])=k_{obs}×t.

### Example 1

### Assay for Prostate Specific Antigen (PSA)

To demonstrate the proof of principle and a potential application, a binding-induced TWJ as a sensor for prostate specific antigen (PSA) in human serum was constructed. Polyclonal anti-PSA antibodies were conjugated to DNA motifs TB and B*C through streptavidin-biotin interactions (FIG. 1A). DNA motifs were modified by labelling T*C* with a fluorescent dye FAM and labelling C with a quencher. Because the FAM-labelled T*C* was initially hybridized with the quencher-labelled C, the fluorescence was quenched. However, in the presence of the target PSA, the binding of PSA to two antibodies brings TB and B*C in close proximity, resulting in the formation of the FAM-labelled binding-induced TWJ (TB:B*C:T*C*) and the simultaneous release of the quencher-labelled C (FIG. 1A). Thus, the binding-induced TWJ becomes fluorescent. By monitoring this fluorescence increase, the amount of the target PSA can be quantified in real-time.

FIG. 1B shows the increases in fluorescence signal from the determination of PSA (0-285 ng/mL) using the binding-induced TWJ. The measured fluorescence increases over a period of 150 min are proportional to the concentrations of PSA ranging from 4.5 to 285 ng/mL (FIG. 1B). A calibration between the fluorescence intensity and the concentration of PSA is linear within the range of concentrations tested (FIG. 1C, red line). An estimated detection limit is 2.8 ng/mL.

Having constructed a binding-induced TWJ sensor for PSA, the ability to detect target proteins in complicated sample matrix, e.g. human serum samples, was explored. PSA was spiked to 10-time diluted human serum, and then the PSA concentrations were quantified using the binding-induced TWJ sensor. As shown in FIG. 1C, similar detection sensitivity for PSA in human serum samples (blue line) as in buffer solutions (red line) was demonstrated, suggesting that their binding-induced TWJ sensor can be applied to real-world sample analysis with no need for any separation. Furthermore, comparing the calibration curve of spiked PSA in serum with that in buffer, slight background increase was observed, and this is mainly due to the background fluorescence from the serum samples. The values of slopes from the two calibration curves are comparable, suggesting that there is minimum matrix effect exist in the serum samples. Such ability to quantify minute amount of PSA (ng/mL level) from human serum samples that contain extremely high concentrations of interference proteins (mg/mL level) without any separation steps suggests that this sensor is very specific to the target protein. Indeed, the use of highly specific PSA antibodies and the principle that assembly and formation of DNA TWJ is triggered only when the affinity binding of two specific antibodies to a single target PSA molecule occurs ensure the high specificity and low background signals from nonspecific interactions.

### Example 2

### Assay for human α-thrombin

Another binding-induced TWJ sensor for the specific detection of human α-thrombin was constructed. Two DNA aptamers that can specifically bind to two distinct binding-epitopes on the same thrombin molecule were used as affinity ligands instead of antibodies (FIG. 2A). As shown in FIG. 2B, a calibration between the fluorescence intensity (background subtracted) and the concentration of thrombin is linear within the range of concentrations tested (50 pM to 30 nM, r2=0.9848). We have also examined the use of biotin as ligands for the detection of streptavidin (FIG. 24), and a linear calibration was also achieved between 50 pM to 10 nM, further demonstrating the versatility of this strategy.

### Example 3

### Design Parameters Influencing the Kinetics

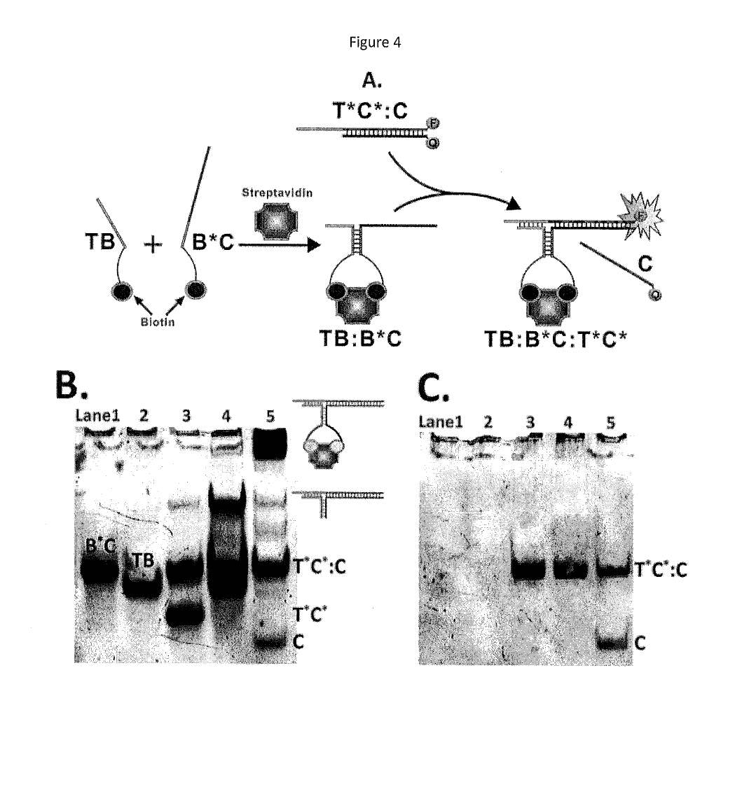

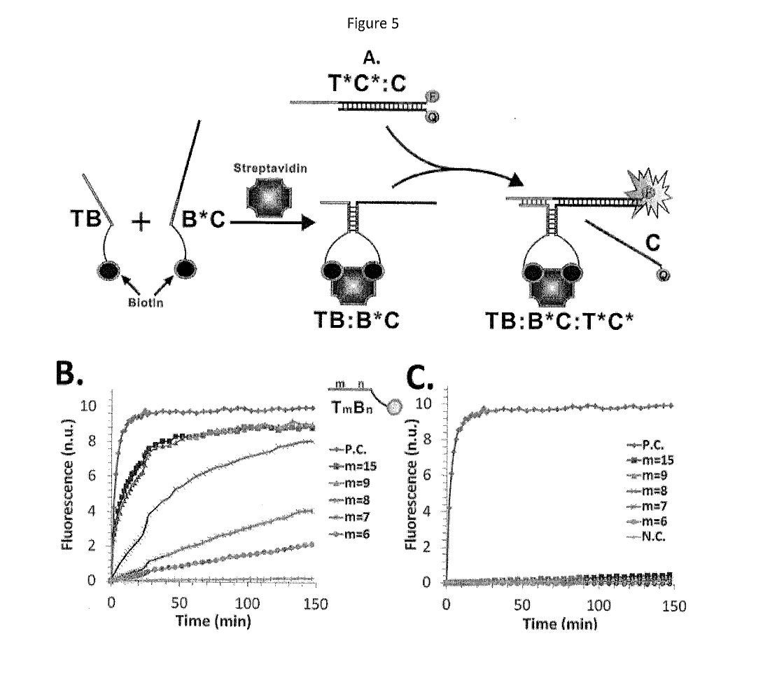

One important element to the success in constructing a real-time sensor for PSA is to achieve a relatively fast DNA strand displacement between TB:B*C and T*C*:C upon the target binding, while minimizing target-independent strand displacement. To fully understand the kinetics of the DNA strand displacement involved in the formation of binding-induced TWJ, streptavidin was used as a target and biotin was used as the affinity ligand to optimize the key reaction parameters (FIG. 4 and FIG. 5).

By monitoring the released quencher-labelled C from T*C*:C, the strand displacement between TC and T*C*:C (FIG. 23) was confirmed. Using gel electrophoresis, the oligonucleotides and their associated products that were involved in the formation of the binding-induced TWJ and the process of strand displacement were characterized. FIG. 4 shows the characterization of relevant oligonucleotides using polyacrylamide gel electrophoresis (PAGE). In the absence of the target streptavidin, the incubation of TB, B*C, T*C*:C, and extra amount of T*C* for 30 min leads to the formation of TB:B*C:T*C* (FIG. 4B, lane 4). There is no observable band corresponding to C, suggesting that there is no release of C and the formed TB:B*C:T*C* was only resulted from bindings among TB, B*C, and extra amount of T*C*. It should be noted here that the use of extra amount of T*C* over C is only to ensure that all FAM labeled C molecules are hybridized with T*C*, and the release of C is only due to the strand displacement reactions. However, in the presence of the target streptavidin (FIG. 4B, lane 5), there is a strong band of the target-induced TWJ at the top of the lane and a clear band of C, indicating the formation of binding-induced TWJ and the strand displacement between TB:B*C and T*C*:C.

In the set of experiments shown in FIG. 24B, all oligonucleotides were detected after the SYBR gold staining. Also conducted was a supplemental set of native PAGE experiments without using staining (FIG. 4C). The motif C was labeled with the fluorescent dye FAM. Fluorescence detection of the gels revealed only the fluorescent C and its associated products. Again, the band of the released C be observed in the gel (FIG. 4C, lane 5) only in the presence of the target streptavidin. These results confirm the strand displacement between TB:B*C and T*C*:C in response to the target binding. Fluorescence was monitored in real-time from the formation of FAM-labelled TWJ product (FIG. 24. The fluorescence response is proportional to the concentration of the target proteins (0.16-10 nM streptavidin).

In an effort to optimize the kinetics involved in the binding-induced TWJ processes, the toehold domain T was designed to have varied lengths from 6 nucleotides (nt) to 9 nt. As shown in FIG. 5B, with the increase of the toehold length from 6 nt to 9 nt, the rate of fluorescence increase is accelerated by 27 times. This substantial enhancement is probably due to the increased kinetics for binding-induced strand displacement between TB:B*C and T*C*:C.¹⁹ Increasing the length of toehold further from 9 nt to 15 nt does not lead to further increase in the reaction rate. Importantly, there is no noticeable background fluorescence signal increase for any of these designs, even after incubation for 150 min (FIG. 5C), suggesting that this strategy is able to maintain an extremely low level of target-independent formation of TWJ.

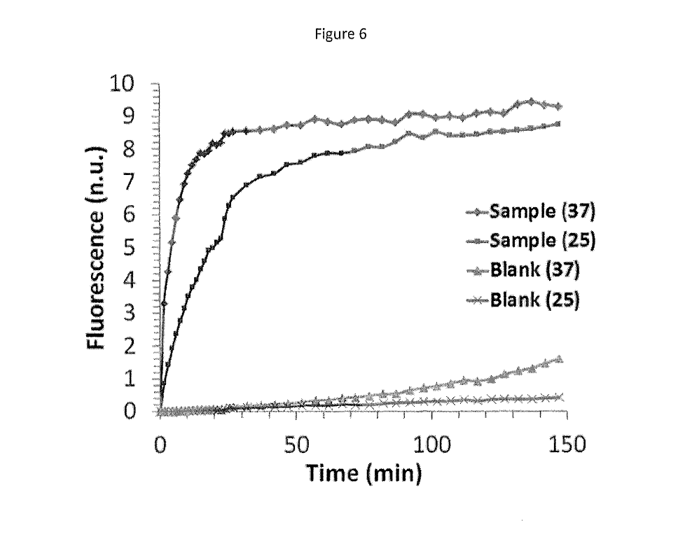

To further maximize the speed of the binding-induced TWJ, the reaction temperature was increased from 25° C. to 37° C. As shown in FIG. 6, the increase in the reaction temperature accelerated the formation of binding-induced TWJ (k_{obs}=1.58×10⁻³ s⁻¹ at 37° C. and k_{obs}=0.60×10⁻³ s⁻¹ at 25° C.). Over 90% fluorescence signal was generated within 10 min. Although target-independent strand displacement between B*C and T*C*:C may be expected to increase with the increase in reaction temperature, these results show that no noticeable background fluorescence increase until after 60 min, providing a time frame long enough for its potential applications, e.g. biomolecular sensing or imaging.

### Example 4

### Comparison with DNA Strand-Displacement Strategies

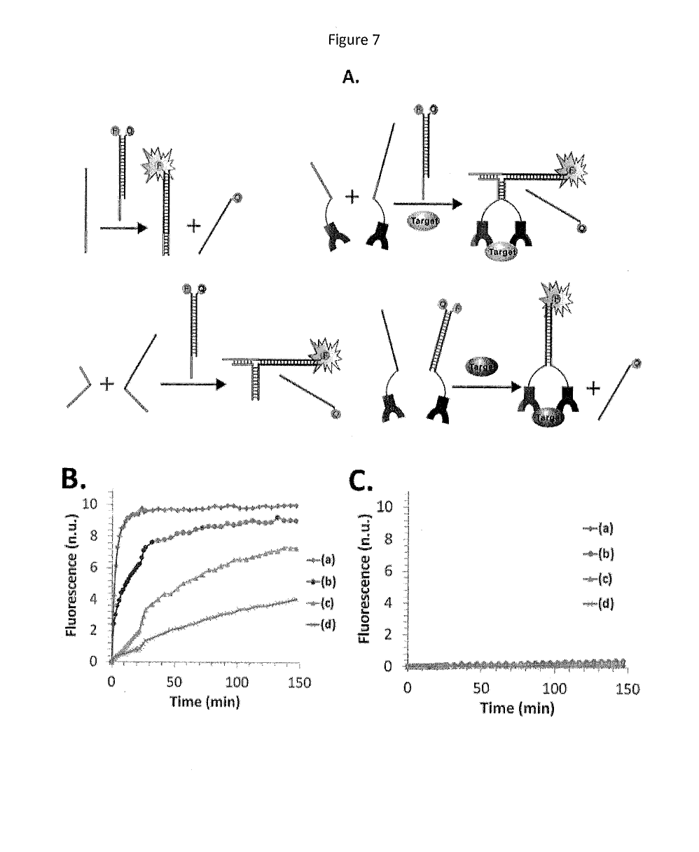

Protein-responsive TWJs may be adapted to existing dynamic DNA assemblies, including DNA logic gates, molecular translators, stepped DNA walkers, and autonomous DNA machines. To explore this potential, technique (b) was compared with three other widely-used DNA strand displacement strategies (FIG. 7), including toehold-mediated DNA strand displacement (a), associative DNA toehold (also known as combinatorial toehold) mediated strand displacement (c), and binding-induced DNA strand displacement (d). For a meaningful comparison, the identical duplex sequences (blue colour) were used for all four techniques. In addition, a, b, and c have the same DNA toehold sequences (red colour). (b) and d have the same linker length (black colour). Results in FIG. 7B show the kinetic profiles of four DNA strand-displacement techniques in the presence of 10 nM target DNA or protein. Comparing to other techniques, this strategy (b) exhibited fast reaction kinetics (ranked as the second fastest displacement reaction in FIG. 7B) and extremely low background from the target-independent displacement (FIG. 7C). The toehold-mediated DNA strand displacement (a) and the associative (combinative) toehold-mediated DNA strand displacement (c) have been successfully used in dynamic DNA assemblies. Having a kinetic profile positioned between those two successful techniques (FIG. 7B), it is predicted that binding-induced TWJ technique (b) can be applied in dynamic DNA assemblies, Importantly, the binding-induced TWJ broadens the scope of dynamic DNA assemblies to beyond DNA and to have the assemblies triggered by protein binding.

Dynamic DNA Assemblies Mediated by Binding-Induced DNA Strand Displacement.

Dynamic DNA assemblies, including catalytic DNA circuits, DNA nanomachines, molecular translators, and reconfigurable nanostructures, have shown promising potential to regulate cell functions, deliver therapeutic reagents, and amplify detection signals for molecular diagnostics and imaging. However, such applications of dynamic DNA assembly systems have been limited to nucleic acids and a few small molecules, due to the limited approaches to trigger the DNA assemblies. Binding-induced DNA strand displacement strategies may convert protein binding to the release of a predesigned output DNA at room temperature with high conversion efficiency and low background. These strategies allow for the construction of DNA assembly systems that are able to respond to specific protein binding, opening an opportunity to initiate dynamic DNA assembly by proteins.

Over the past 30 years, tremendous effort has contributed to the successful development of DNA nanostructures and nanodevices. Attention has recently shifted from designing DNA nanostructures/devices to exploring their potential functions in biological systems, including regulating cell activities, delivering therapeutic compounds, and amplifying detection signals. Successful applications of DNA assembly systems have been limited to nucleic acids and a few small molecules. It remains a challenge to apply DNA assembly systems to respond to specific proteins. Applicants have developed a binding-induced DNA strand displacement strategy that uses proteins to initiate the process of diverse dynamic DNA assemblies.

Toehold-mediated strand displacement is currently the most widely used strategy to direct dynamic DNA assemblies. The binding-induced DNA strand displacement strategy described herein relies on protein binding to accelerate the rates of strand displacement reactions. Thus, the specific protein initiates the strand displacement process, and the displaced output DNA triggers dynamic DNA assemblies. To demonstrate this principle, an isothermal binding-induced DNA strand displacement strategy is shown that is able to release the predesigned output DNA at room temperature with high conversion efficiency and low background. Then, this strategy is applied to design two dynamic DNA assembly systems that are triggered by protein binding: a binding-induced DNA strand displacement beacon and a binding-induced DNA circuit.

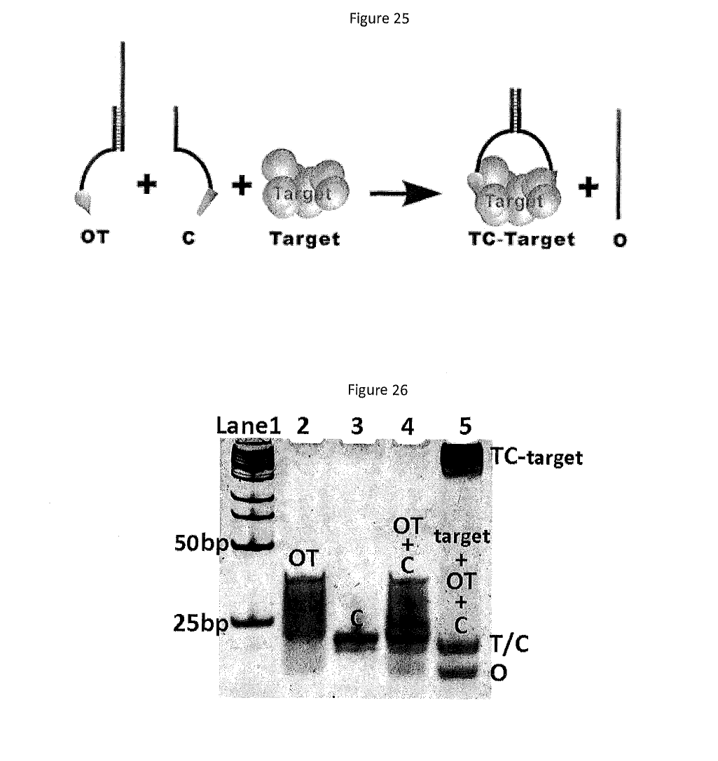

The strategy is illustrated schematically in FIG. 25. The binding-induced strand displacement strategy is designed to have target recognition and signal output elements. Target recognition is achieved by two specific affinity ligands binding to the same target molecule. One affinity ligand is conjugated to the output DNA motif (OT) that is formed by prehybridizing the output DNA (O) and the support DNA (T), and the other is conjugated to the competing DNA motif (C). The complementary sequence of OT was designed to have the same length as C. Thus, in the absence of the target molecule, the rate of the strand exchange reaction between OT and C is extremely slow at 25° C. However, in the presence of the target molecule, binding of the target molecule to the two affinity ligands that are linked to OT and C brings C in close proximity to OT. This process greatly increases the local concentration of C and accelerates the strand displacement reaction between OT and C. As a consequence, the output DNA O is released from its support T. The subsequent dynamic DNA assembly can be triggered by O, e.g., using the principle of toehold-mediated strand displacement. To be more specific, the toehold part of O is designed to be embedded in the complementary part of OT (FIG. 25 black), so no dynamic DNA assembly can be triggered unless the target molecule is present and the toehold part of the output DNA is released.

Materials and Reagents

Streptavidin from Streptomyces avidinii (product number, S4762), biotin (product number, B4501), bovine serum albumin (BSA), magnesium chloride hexahydrate (MgCl2.6H2O), and 100× Tris-EDTA (TE, pH 7.4) buffer were purchased from Sigma. SYBR Gold and ROX Reference Dye (ROX) were purchased from Invitrogen. Reagents for polyacrylamide gel electrophoresis (PAGE), including 40% acrylamide mix solution and ammonium persulfate were purchased from BioRad Laboratories (Mississauga, ON, Canada). Low molecular DNA ladder was purchased from New England Biolabs. Tween 20 and 1, 2-bis(dimethylamino)-ethane (TEMED) were purchased from Fisher Scientific (Nepean, ON, Canada). NANOpure H₂O (>18.0 MΩ), purified using an Ultrapure Milli-Q water system, was used for all experiments. All DNA samples were purchased from Integrated DNA Technologies (Coralville, Iowa) and purified by HPLC. The DNA sequences and modifications are listed in Table 4:

TABLE 4
DNA sequences and modifications used to construct binding-induced DNA
strand displacement beacons and catalytic DNA circuits for streptavidin.

| | Name | Sequences |
|---|---|---|
| For binding- | O | 5′-ATA GAT CCT CAT AGC GAG ACC TAG CAA-3′ |
| induced DNA | | [SEQ ID NO: 17] |
| strand | L | 5′-TT AGT CCT ACA GCA GTA ACG ACT ATA GAT CCT CAT AGC |
| displacement | | GAG ACC TAG CAA-3′ |
| and | | [SEQ ID NO: 18] |
| displacement | T (12 nt) | 5′-biotin-TTT TTT TTT TTT TTT TTG CTA GGT CTC-3′ |
| beacon | | [SEQ ID NO: 19] |
| | T (14 nt) | 5′-biotin-TTT TTT TTT TTT TTT TTG CTA GGT CTC GC-3′ |
| | | [SEQ ID NO: 20] |
| | T (16 nt) | 5′-biotin-TTT TTT TTT TTT TTT TTG CTA GGT CTC GCT A-3′ |
| | | [SEQ ID NO: 21] |
| | T (18 nt) | 5′-biotin-TTT TTT TTT TTT TTT TTG CTA GGT CTC GCT ATG-3′ |
| | | [SEQ ID NO: 22) |
| | T (20 nt) | 5′-biotin-TTT TTT TTT TTT TTT TTG CTA GGT CTC GCT ATG AG-3′ |
| | | [SEQ ID NO: 23] |
| | C (12 nt) | 5′-GAG ACC TAG CAA TTT TTT TTT TTT TTT-biotin-3′ |
| | | [SEQ ID NO: 24] |
| | C (14 nt) | 5′-GC GAG ACC TAG CAA TTT TTT TTT TTT TTT-biotin-3′ |
| | | [SEQ ID NO: 25) |
| | C (16 nt) | 5′-T AGC GAG ACC TAG CAA TTT TTT TTT TTT TTT-biotin-3′ |
| | | [SEQ ID NO: 26) |
| | F | 5′-FAM-ATA GAT CCT CAT AGC GAG AC-3′ |
| | | [SEQ ID NO: 27] |
| | Q | 5′-TTG CTA GGT CTC GCT ATG AGG ATC TAT-Dabcyl-3′ |
| | | [SEQ ID NO: 28] |
| For | O | 5′-A TAGATCCT CATAGCGA GACCTAG CCA |
| binding- | | [SEQ ID NO: 29] |
| induced | H1 | 5′-CTAGGTC TCGCTATG AGGATCTA CCATCGTGTAC TAGATCCT |
| catalytic | | CATAGCGA AAGAGCAC CCTTGTCA-3′ |
| DNA circuit | | [SEQ ID NO: 30] |
| | H2 | 5′-AGGATCTA GTACACGATGG TAGATCCT CATAGCGA CCATCGTGTAC-3′ |
| | | [SEQ ID NO: 31] |
| | F′ | 5′-FAM-TGACAAGG GTGCTCTT TCGCTATG-3′ |
| | | [SEQ ID NO: 32] |
| | Q′ | 5′-AAGAGCAC CCTTGTCA-Dabcyl |
| | | [SEQ ID NO: 33] |
| | P | 5′-CATAGCGA AAGAGCAC CCTTGTCA-3′ |
| | | [SEQ ID NO: 34] |

Probe Preparation for Binding-Induced DNA Strand Displacement

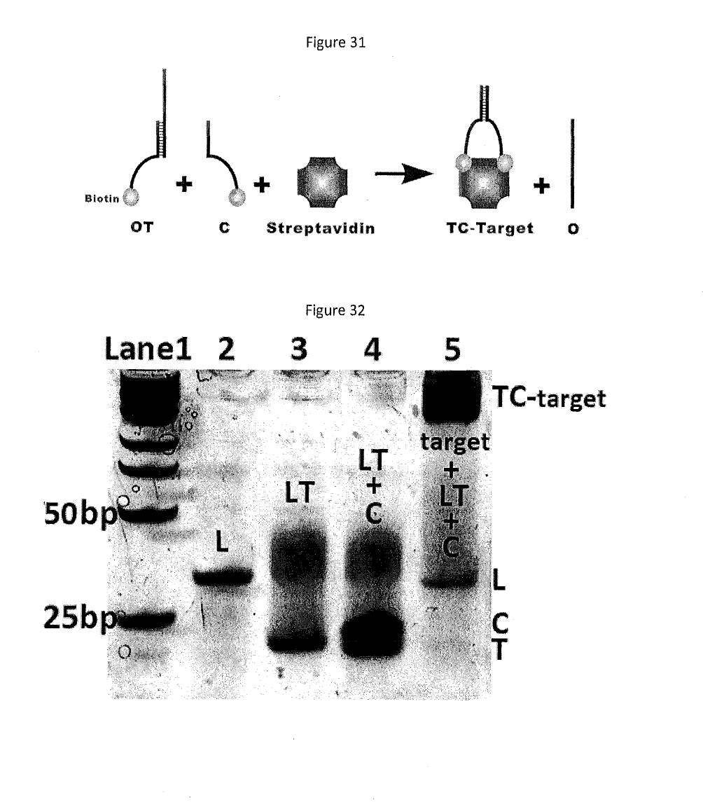

The binding-induced DNA strand displacement strategy for streptavidin is schematically shown in FIG. 31. DNA probe (OT) for binding-induced strand displacement was prepared at a final concentration of 5 μM by mixing 20 μM 50 μM supporting DNA (T) with 13.3 μL 50 μM Output DNA (O) in 166.7 μM TE-Mg (1×TE, 10 mM MgCl2, 0.05% Tween20) buffer, heating to 90° C. for 5 min, and allowing solution to cool down to 25° C. slowly in a period of 3 hours, Probe (FQ) for displacement beacon was also prepared at a final concentration of 5 μM by mixing 20 μM 50 μM FAM labeled DNA (F) with 20 μM 50 μM dark quencher labeled DNA (Q) in 160 μM TE-Mg buffer, heating to 90° C. for 5 min, and allowing solution to cool down to 25° C. slowly in a period of 3 hours. Reporter (F′Q′) for catalytic DNA circuit was prepared the same way as FQ, except that the ratio between F′ and Q′ was kept to 1:2 to minimize the background fluorescence.

Monitor the Binding-Induced DNA Strand Displacement Using Gel Electrophoresis

For a typical binding-induced DNA strand displacement reaction, the reaction mixture contained 2 μM probe OT, 2 μM competing DNA (C), 1 μM target protein, and TE-Mg buffer. The reaction mixture was incubated at 25° C. for 45 min. After incubation, the performance of binding-induced DNA strand displacement was then assessed using 15% native polyacrylamide gel electrophoresis (PAGE). All the gels were freshly prepared in house. Before loading, DNA samples were mixed with DNA loading buffer on a volume ratio of 5:1. A potential of 12 V/cm was applied for gel electrophoresis separation. After separation, PAGE gels containing DNA were stained using SYBR gold, and imaged by ImageQuant 350 (IQ350) digital imaging system (GE Healthcare).

Binding-Induced DNA Strand Displacement Beacon

For a typical binding-induced DNA strand displacement beacon, the reaction mixture contained 10 nM probe OT, 10 nM competing DNA (C), 20 nM displacement beacon FQ, 50 nM ROX, 1 μM polyT oligo, varying concentrations of the target protein, and TE-Mg buffer. The reaction mixture was incubated at 25° C. for 45 min in a 96-well plate. Fluorescence was measured directly from the microplate using a multi-mode microplate reader (DX880, Beckman Coulter) with both excitation/emission at 485/515 nm for displacement beacon and excitation/emission at 535/595 nm for ROX as a reference dye. The measured fluorescent signal was normalized so that 1 n.u. of fluorescence corresponded to fluorescent signal generated by 1 nM O. This normalization was achieved using a positive control containing 10 nM 0, 20 nM FQ, 1 μM polyT oligo, and 50 nM Rox in TE-Mg buffer, and a negative control containing identical reagents in positive control except that there was no O added. To monitor the kinetic process of binding-induced DNA strand-displacement, fluorescence of the reaction mixture was collected every 1.5 minutes for the first 30 minutes and then every 5 minutes for another 2 hours.

Estimation of the Conversion Efficiency of the Binding-Induced DNA Strand Displacement Beacon

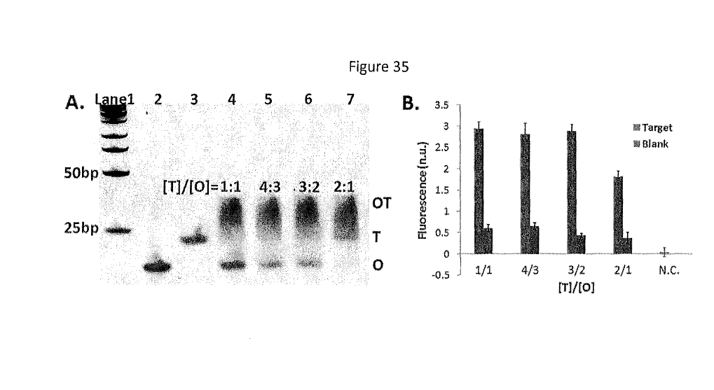

The conversion efficiency was calculated as ratios of the experimentally determined concentrations of O over their theoretical values. The experimentally determined concentrations of O were achieved by normalizing fluorescence intensities against different controls (details in the previous section in the supporting information). The theoretical concentrations of O were calculated based on the probability of each streptavidin to form the OTC-Target binding complex. (FIG. 34) Briefly, as the probability for each streptavidin molecule to form an effective OTC-Target complex is 1.25 (FIG. 34), and each OTC-Target complex yields ⅔ output DNA O on average ([O]/[T] was optimized to be ⅔, FIG. 35), thus the theoretical concentration of O equal to [target]×1.25×⅔.

Binding-Induced Strand-Displacement Beacon for the Detection of PDGF-BB

A DNA aptamer (Apt) for the homodimer BB of platelet derived growth factor (PDGF-BB) was linked to OT and C, forming Apt-OT and Apt-C probes (Table 5). These probes were used to develop a binding-induced DNA strand-displacement beacon for the detection of PDGF-BB,

TABLE 5
DNA sequences and modifications used to devise a binding-
induced DNA strand-displacementbeacon for the detection
of homodimer of platelet derived growth factor (PDGF-BB).

| DNA name | | |
|---|---|---|
| Binding- | O | 5′-ATA GAT CCT CAT AGC GAG ACC TAG CAA-3′ |
| induced | Apt-T | [SEQ ID NO: 35) |
| DNA | (12 nt)* | 5′-TACT CAG GGC ACT GCA AGC AAT TGT GGT CCC |
| strand | | AAT GGG CTG AGTA-TTT TTT TTT TTT TTT TTT TTT TTT |
| | | TTG CTA GGT CTC-3′ |
| | | [SEQ ID NO: 36) |

A reaction mixture containing 20 nM probe Apt-OT, 20 nM competing DNA (Apt-C), 50 nM ROX, 1 μM polyT oligo, varying concentrations of the target PDGF-BB, and TE-Mg buffer was incubated at 37° C. for 15 min. DNA probe FQ was then added to this mixture at a final concentration of 20 nM. After incubating the reaction mixture at room temperature for another 30 min, fluorescence was measured using a multi-mode microplate reader (DX880, Beckman Coulter) with both excitation/emission at 485/515 nm for displacement beacon and excitation/emission at 535/595 nm for ROX reference dye. The measured fluorescent signal was normalized so that 1 normalized unit (n.u.) of fluorescence corresponded to fluorescent signal generated by 1 nM O. This normalization was achieved using a positive control containing 10 nM 0, 20 nM FQ, 1 μM polyT oligo, and 50 nM ROX in TE-Mg buffer, and a negative control containing all reagents as in positive control except that there was no O added.

Elimination of Target-Independent Displacement

To examine the target-independent displacement, the use of OT and C of different lengths was tested, from 12 nt to 20 nt. Applicants found that a shorter C (12 nt) than OT (14-20 nt) was appropriate because the shorter competing DNA could not readily displace the longer output DNA O. As long as OT was longer than C by 2 nt or more, the target-independent displacement could be substantially reduced or eliminated. To maximize the signal-to-background ratio, 14 nt for OT and 12 nt for C were chosen.

Binding-Induced Catalytic DNA Circuit

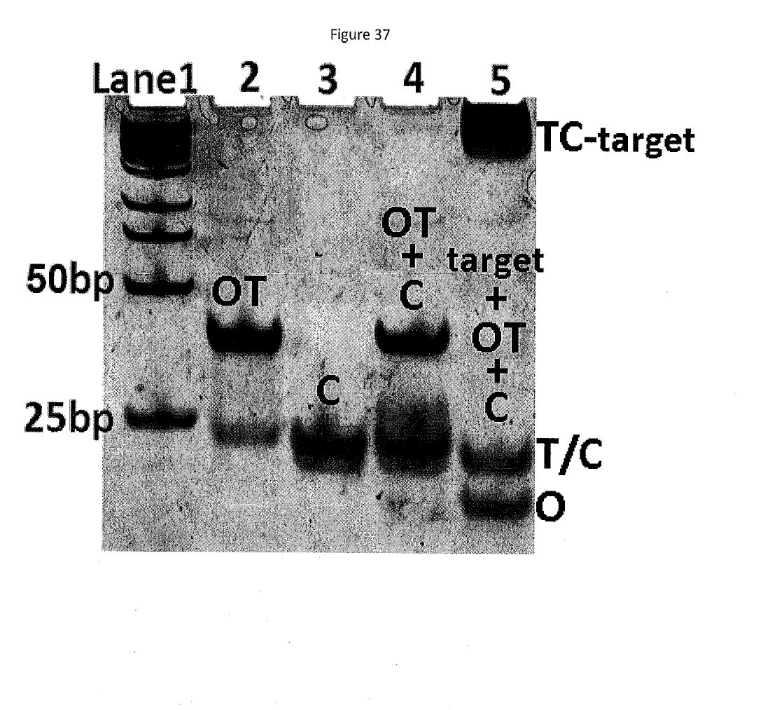

For a typical binding-induced catalytic DNA circuit, the reaction mixture contained 125 nM H1, 200 nM H2, 125 nM F′Q′, 20 nM OT, 20 nM C, 1 μM polyT oligo, 50 nM ROX, varying concentrations of target protein, and TE-Mg buffer. The reaction mixture was incubated at 25° C. in 96-microplate well, and fluorescence was monitored directly from the multimode microplate reader. To monitor the reaction at real-time, fluorescent signal was collected every 1.5 minutes for the first 30 minutes and then every 5 minutes for another 3.5 hours. To normalize the fluorescent signal, both positive and negative controls were used (FIG. 37). A positive DNA (P) was designed to be able to trigger the reporter (F′Q′) independently from the catalytic DNA circuit, and thus could be used to serve as a positive control to normalize the fluorescence intensities generated by the binding-induced catalytic DNA circuit. The positive control contained 50 nM P for reporter F′Q′, 125 nM H1, 200 nM H2, 125 nM F′Q′, 1 μM polyT oligo, 50 nM ROX, and TE-Mg buffer. The negative control contained the identical reagents in the positive control, except that there was no P added.

One binding-induced strand displacement strategy for streptavidin used biotin as the affinity ligand (FIG. 31). Streptavidin was selected for its extremely high binding affinity to biotin (Kd=10⁻¹⁴ M). This strong interaction ensures that the target binding process will not limit the performance of the binding-induced strand displacement. T and C were each conjugated with a biotin molecule. The output O was designed to hybridize to T with a complementary length of 12 nt. O was extended with another 15 nt to help direct further DNA assemblies.

FIG. 26 shows the characterization of the relevant oligonucleotides using polyacrylamide gel electrophoresis (PAGE). In the absence of the target streptavidin, the incubation of the two probes OT and C for 45 min does not lead to the release of O (FIG. 26, lane 4), indicating that the rate of strand exchange between OT and C was extremely slow. However, in the presence of streptavidin, the observed strong bands of O and TC-target complex indicate the release of O from OT and the formation of TC-target complex according to FIG. 25. These results suggest that the binding between streptavidin and biotin accelerated the kinetics of strand displacement reaction between OT and C.

As many dynamic DNA assembly systems, e.g., DNA catalytic circuits and nanomachines, use longer DNA molecules (e.g., 50 nt), the versatility of the strategy to output DNA of 50 nt (L) in length was further tested. As shown in FIG. 32, a strong band of L appeared in lane 5 upon target binding, indicating that our strategy is applicable to release diverse output DNA molecules. Having achieved isothermal binding-induced strand displacement, Applicants further show that this strategy is able to direct dynamic DNA assemblies, using two examples: a strand displacement beacon and a catalytic DNA circuit.

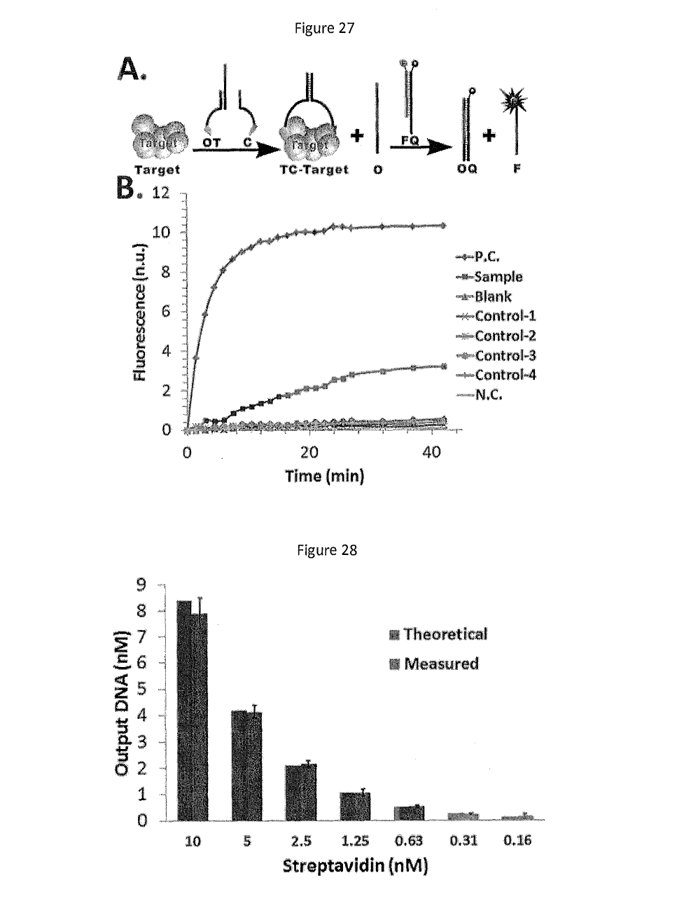

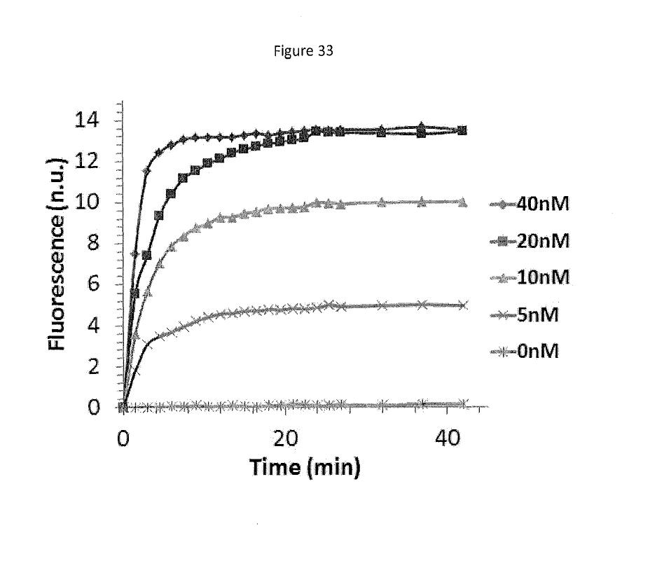

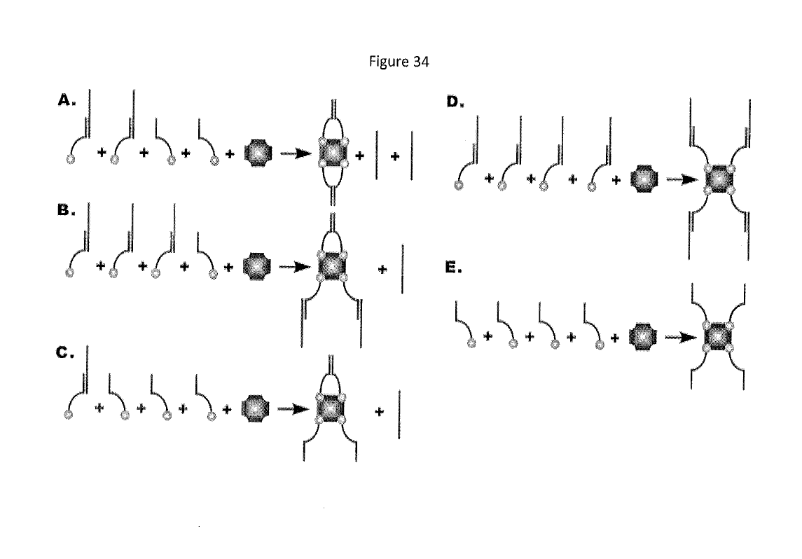

Applicants' first designed a toehold-mediated strand displacement beacon that was able to respond to the output DNA O (FIG. 27A). Briefly, two complementary DNA strands are labeled with a fluorophore (F) and a quencher (Q), respectively. Q is designed to have 7 nt longer than F, which serves as a “toehold” for the hybridization of Q to the output DNA O. In the absence of O, a stable DNA duplex is formed between F and Q, and the fluorescence signal is quenched. However, in the presence of O, the toehold-mediated strand displacement reaction is initiated and F is released from Q, turning on the fluorescence signal (FIG. 33). Thus, the binding-induced displacement beacon can be used to determine protein binding through monitoring of the displaced O.

FIG. 27B shows the fluorescence signal increase of the binding-induced displacement beacon for streptavidin as a function of time. Within a period of 45 min, fluorescence intensities from 10 nM streptavidin (red curve) are readily distinguishable from the blank (green curve) that contained all reagents but not the target streptavidin. To confirm that the binding-induced displacement beacon is target specific, Applicants tested our system using the same 10 nM streptavidin that was fully saturated with 500 μM of free biotin (Control-1). The results are similar to those of the blank. Likewise, in the absence of O (Control-2), C (Control-3), or OT (Control-4), only back-ground fluorescence was detectable. These results suggest that specific binding is responsible for the fluorescence signals from the binding-induced displacement beacon.

Having established the binding-induced displacement beacon, Applicants further estimated its efficiency of converting target streptavidin to the output DNA O (FIG. 34) at different target concentrations. By comparing the experimentally determined concentrations of O with their theoretical concentrations, Applicants found that the average converting efficiency was 99.3±7.6% throughout a wide range of target concentrations (160 pM to 10 nM) (FIG. 28).

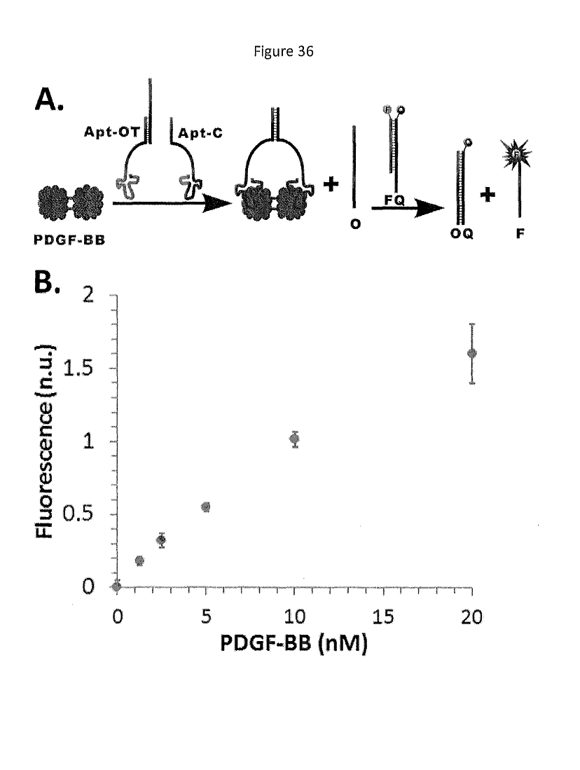

The binding-induced displacement beacon strategy was applied to the analysis of an example of a clinically relevant protein, platelet derived growth factor (PDGF). A DNA aptamer for PDGF-BB was incorporated into the DNA probes OT and C, forming Apt-OT and Apt-C(Table 5). Binding of PDGF-BB to its aptamer sequences in OT and C brought the two DNA probes together, resulting in the displacement of output DNA O (FIG. 36A). The released output DNA O triggered a subsequent toehold-mediated strand displacement reaction, releasing F from FQ. Fluorescence intensity from F provided a measure for the detection of PDGF-BB. The fluorescence intensity increases with the increase of PDGF concentration (FIG. 36B). These quantitative results demonstrate an application of the binding-induced strand displacement beacon to the detection of PDGF protein.

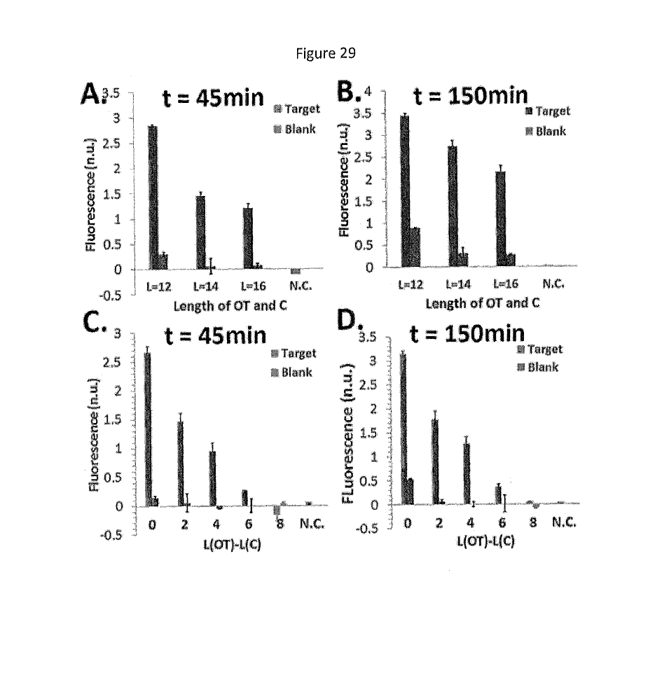

The success of binding-induced displacement beacon opens up opportunities for directing further dynamic DNA assemblies, e.g., catalytic DNA circuit. Because these DNA assemblies of higher structural complexity often require extended periods of incubation, it is critical to minimize the background that can also be amplified over the extended periods (FIG. 29B). Thus, Applicants optimized the designs of oligonucleotides, OT and C, to minimize target-independent strand displacement. This optimization is based on the previous discovery that increasing the length of DNA duplex could slow down the rate of strand exchange reactions drastically.

As shown in FIGS. 29A and B, in the presence of 10 nM streptavidin, the fluorescence intensities decrease with increasing length of OT and C from 12 to 16 nt. An extended incubation period (e.g., 150 min) results in noticeable increases in background (FIG. 29B), suggesting the target-independent displacement of output DNA O. To eliminate the target independent displacement, Applicants' fixed the competing DNA C to be 12 nt in length, and increased the length of OT from 12 to 20 nt. In principle, shorter competing DNA is thermodynamically unfavored to displace a longer DNA strand, and thus should be able to suppress nonspecific release of O. Indeed, FIGS. 29C and D shows that the nonspecific displacement can be eliminated even after incubation for 150 min. To maximize signal-to-background, a 2-nt difference between OT (14 nt) and C (12 nt) was chosen. This optimized condition was also examined with PAGE (FIG. 37), and no output DNA O band was observed on the gel without target molecule (lane 4), while a strong O band appeared with target (lane 5). These results confirm that Applicants methods are able to eliminate the target-independent displacement of output DNA O.

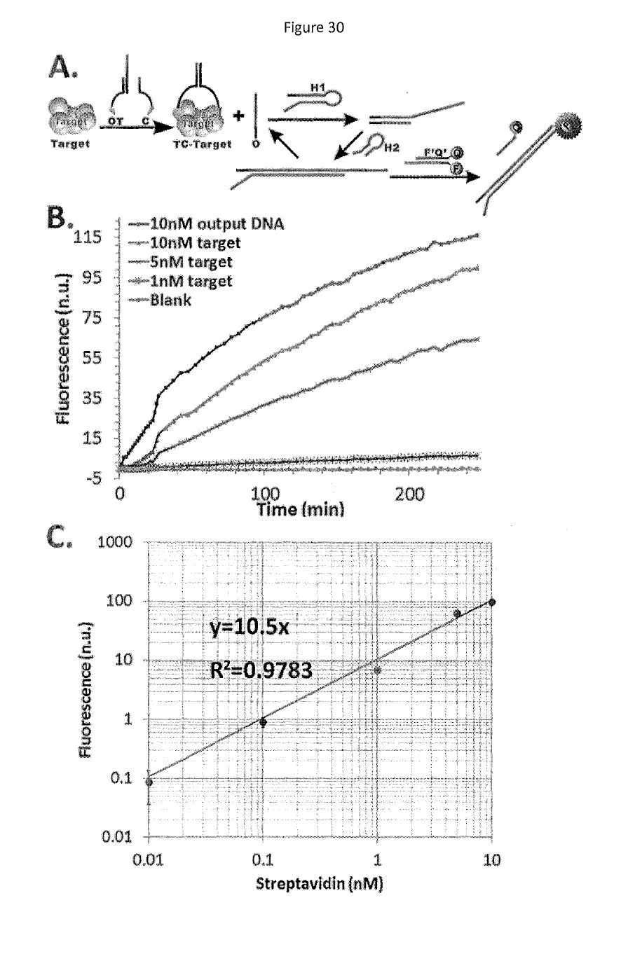

Upon eliminating the target-independent displacement, a binding-induced catalytic DNA circuit was designed to demonstrate the ability of our strategy to direct dynamic DNA assemblies with higher structural complexity. The principle of our binding-induced catalytic DNA circuit strategy is shown in FIG. 30 a pair of DNA hairpins (H1 and H2) is designed to partially hybridize to each other. However, the spontaneous hybridization between H1 and H2 is kinetically hindered by caging complementary regions in the stems of the hairpins. In the presence of the target molecule, the output DNA O is released by the binding-induced strand displacement reaction. The released output DNA opens the stem part of H1 by the principle of the toehold-mediated DNA strand displacement. The newly exposed sticky end of H1 nucleates at the sticky end of H2 and triggers another strand-displacement reaction to release O. Thus, O is able to act as a catalyst to trigger the formation of other H1-H2 complexes. This process results in amplification of the detection signals.

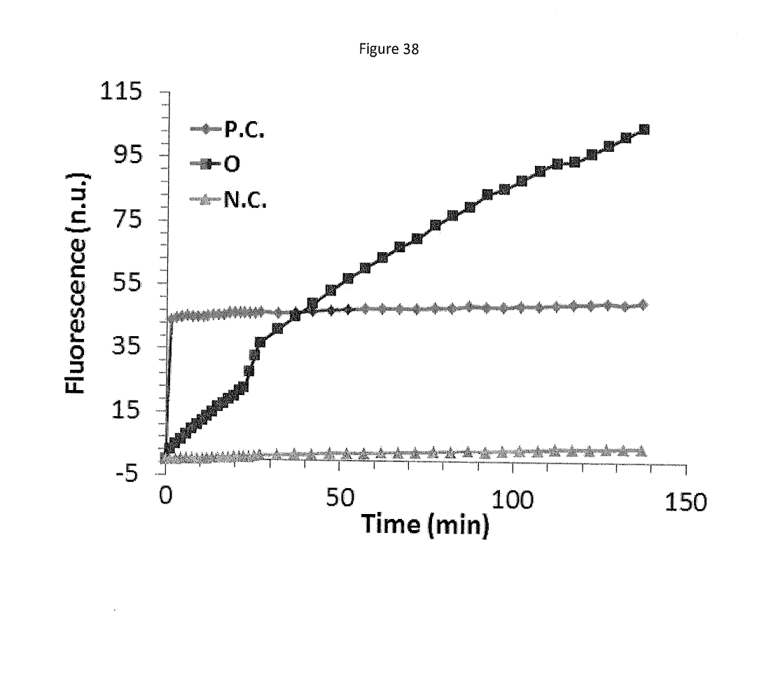

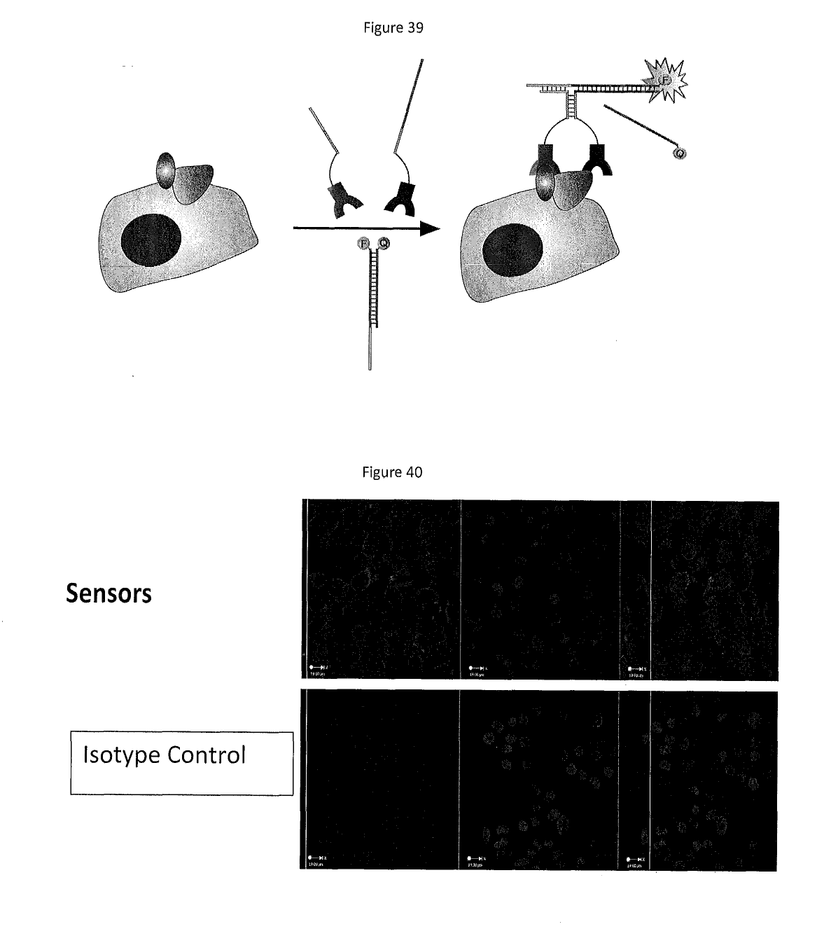

To test the signal amplification ability of our binding-induced DNA circuit, Applicants monitored the fluorescence intensity increase as a function of time over a period of 4 h. As shown in FIG. 30B, the fluorescence intensity generated from 10 nM streptavidin is close to 100 normalized units, which corresponds to 100 nM positive DNA (P) (FIG. 38). Essentially no background fluorescence signal was observed for the blank. Compared to the toehold-mediated catalytic DNA circuit that is triggered directly by the output DNA O (FIG. 30B, red curve), the binding-induced catalytic DNA circuit (FIG. 30B, green curve) demonstrates comparable signal amplification capability. Furthermore, the measured fluorescence intensities are responsive to the concentrations of streptavidin in the range of 10 pM to 10 nM (FIG. 30C), demonstrating the capability for quantification. Applicants estimated from the standard curve (FIG. 30C) that the fluorescence signal has been amplified by over 10-fold throughout this concentration range.

Real-Time Cell Surface Sensing

To achieve real-time cell surface sensing, polyclonal anti-HER2 antibodies (goat IgG) were conjugated to DNA probes through streptavidin-biotin conjugation. DNA probes designs were the same as previously described for binding-induced TWJ sensors. For imaging HER2 from cell surface, SK-BR-3 breast cancer cells expressing HER2 were seeded into a 35-mm glass bottom culture at a concentration of 5×10⁴ cells per well. When culturing to 90% confluence, cells were fixed with 4% paraformaldehyde for 30 min. After permeabilizing fixed cells for 10 min using PBST buffer, binding-induced DNA TWJ sensor components, including 25 nM anti-HER2 antibody-conjugated DNA probe pairs, 50 nM strand displacement beacons (FQ), and 100 nM DAPI were added to the culture dish. After adding all sensor components, cells can be directly observed under confocal fluorescence microscope without any washing steps (FIG. 40). Fluorescence imaging of fixed cells was performed on an Olympus IX-81 microscope that was coupled with a Yokagawa CSU X 1 spinning disk confocal scan-head and Hamamatsu EMCCD cameras with a 20×/0.85 Oil objective lens. A 405 nm pumped diode laser was used for the excitation of nucleus staining dye DAPI. A 491 nm pumped diode-pumped laser was used for the excitation of FAM labeled binding-induced TWJ sensor. The exposure time was set to be 200 ms for DAPI and 600 ms for FAM-labeled sensors for both sample (top row) and isotype control (bottom row). The isotype control was prepared and treated using the same condition as for the sample, except that non-specific goat IgG was used to modify DNA probes instead of HER2-specific goat IgG.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

### REFERENCES

The following references are incorporated by reference herein, where permitted, as though reproduced herein in their entirety.
(1) Seeman, N. C. J Theor. Biol. 1982, 99, 237-247.
(2) Seeman, N. C.; Kallenbach, N. R. Annu. Rev, Biophys. Biomol. Struct. 1994, 23, 53-86.
(3) Goodman, R. P.; Schaap, I. A. T.; Tardin, C. F.; et al. Science, 2005, 310, 1661-1665.
(4) Yang, H.; Sleiman, H. F. Angew. Chem. Int. Ed. 2008, 47, 2443-2446.
(5) Boer, D. R.; Kerckhoffs, J. M. C. A.; Parajo, Y.; et al. Angew. Chem. Int. Ed. 2010, 49, 2336-2339.
(6) He, Y.; Chen, Y.; Liu, H.; Ribbe, A. E.; Mao, C. J. Am. Chem. Soc. 2005, 127, 12202-12203.
(7) Shu, D.; Shu, Y.; Hague, F.; Abdelmawla, S.; Guo, P. Nat. Nanotechnol. 2011, 6, 658-667.
(8) Muller, J.; Lippert, B. Angew. Chem. Int, Ed. 2006, 45, 2503-2505.
(9) Hamada, S.; Murata, S. Angew. Chem. Int, Ed. 2009, 48, 6820-6823.
(10) Thomas, J. M.; Chakraborty, B.; Sen, D.; Yu, H.-Z. J. Am. Chem. Soc. 2012, 134, 13823-13833.
(11) Seemann, I. T.; Singh, V.; Azarkh, M.; Drescher, M.; Hartig, J. S. J. Am. Chem. Soc. 2011, 133, 4706-4709.
(12) Thomas, J. M.; Yu, H.-Z.; Sen, D. J. Am. Chem. Soc. 2012, 134, 13738-13748.
(13) Ge, B.; Huang, Y. C.; Sen, D.; Yu, H.-Z. Angew. Chem. Int, Ed. 2010, 49, 9965-9967.
(14) Nutiu, R.; Li, Y. J. Am. Chem. Soc. 2003, 125, 4771-4778.
(15) Chen, X. J. Am. Chem. Soc. 2012, 134, 263-271.
(16) Li, B.; Jiang, Y.; Chen, X.; Ellington, A. D. J Am, Chem. Soc, 2012, 134, 13918-13921.
(17) Genot, A. J.; Bath, J.; Turberfield, A. J. Angew. Chem, Int, Ed, 2013, 52, 1189-1192.
(18) Li, F.; Zhang, H.; Wang, Z.; Li, X.; Li, X.-F.; Le, X. C. J. Am. Chem, Soc. 2013, 135, 2443-2446.
(19) Zhang, D. Y.; Winfree, E. J. Am. Chem, Soc. 2009, 131, 17303-17314.
(20) Seelig, G.; Soloveichik, D.; Zhang, D. Y.; Winfree, E. Science 2006, 314, 1585-1588;
(21) Qian, L.; Winfree, E. Science 2011, 332, 1196-1201.
(22) Liao, S.; Seeman, N. C. Science 2004, 306, 2072-2074.
(23) Picuri, J. M.; Frezza, B. M.; Chadiri, M. R. J. Am. Chem. Soc, 2009, 131, 9368-9377.
(24) Li, F.; Zhang, H.; Lai, C.; Li, X.-F.; Le, X. C. Angew. Chem. Int, Ed. 2012, 51, 9317-9320.
(25) Shin, J.-S.; Pierce, N. A. J. Am. Chem. Soc. 2004, 126, 10834-10835.
(26) Gu, H.; Chao, J.; Xiao, S.-J.; Seeman, N. C. Nature, 2010, 465, 202-205.
(27) He, Y.; Liu, D. R. Nat, Nanotechnol, 2010, 5, 778-782.
(28) Bath, J.; Turberfield, A. J. Nat. Nanotechnol, 2007, 2, 275-284.
(29) Li, F.; Lin, Y.; Le, X. C. Anal. Chem., 2013, 85 (22), pp 10835-10841.
(30) Zhang, H., et al. Chem. Rev. 113, 2812-2841 (2013).
(31) Zhang, H., Li, X.-F., Le, X. C. Anal. Chem 84, 877-884 (2012).
(32) Nutiu R., Li, Y. J. Am. Chem. Soc. 125, 4771-4778 (2003).
(33) Fredriksson, S., et al. Nat. Biotechnol, 20, 473-477 (2002).
(34) Soderberg, et al. Nat. Methods. 3, 995-1000 (2006)
(35) Sokol, D. L., et al. Proc. Natl. Acad. Sci. USA. 95, 11538-11543 (1998)

What is claimed is:

1. A method of detecting a non-DNA target, comprising:
(a) providing a first nucleic acid motif linked to a first affinity ligand which binds specifically to the target and having a first toehold domain and a first binding domain, and a second nucleic acid motif linked to a second affinity ligand which binds specifically to the target and having a second binding domain, wherein the first and second binding domains are complementary to each other;
(b) contacting the target with the first and second nucleic acid motifs to form a target-ligand complex, wherein the formation of the complex causes displacement of an output nucleic acid motif.

2. The method of claim 1 wherein each of the first, second and output nucleic acid motifs comprise DNA.

3. The method of claim 1 wherein the first and second binding domains do not form a stable duplex at room temperature without the presence of the target.

4. The method of claim 2 wherein the output DNA motif is hybridized to one of the first or second DNA motifs, and is displaced by the formation of the complex.

5. The method of claim 2 further comprising the step of contacting the target-ligand complex with a detection probe comprising the output DNA motif, which is displaced after contacting the target-ligand complex.

6. The method of claim 5 wherein the detection probe comprises a second toehold domain complementary to the first toehold domain, and a displacement domain complementary to a displacement domain of the second DNA motif, wherein hybridization of the detection probe to the target-ligand complex displaces the output DNA motif.

7. The method of claim 1 wherein the target is a protein.

8. The method of claim 7 wherein the first and second affinity ligands are the same or different, and at least one is an antibody or an aptamer.

9. The method of claim 2 further comprising the use of a displacement beacon which provides a detectable signal upon displacement of the output DNA motif.

10. The method of claim 5 wherein the displacement beacon comprises a fluorophore carried on the detection probe.

11. The method of claim 10 wherein the detection probe comprises a fluorophore and a quencher, wherein the quencher is linked to the output DNA motif.

12. The method of claim 11 wherein the quencher is a dark quencher.

13. The method of claim 1 wherein the first toehold domain comprises 6, 7, 8 or 9 nucleotides and/or the first and second binding domains each comprise 6 complementary nucleotides.

14. The method of claim 2 wherein the output DNA motif is used in a catalytic DNA circuit and/or a dynamic DNA assembly method.

15. The method of claim 1, adapted to detect an antigen in a biological sample or on the surface of a cell.

16. The method of claim 1, adapted to operate without heat cycling.

17. The method of claim 1, adapted to operate without the use of enzymes.

18. The method of claim 1, adapted to operate as an imaging method, a diagnostic method, or a point-of-care diagnostic method.

19. A protein-DNA three way junction complex comprising a first DNA motif linked to a first affinity ligand bound specifically to the protein, a second DNA motif linked to a second affinity ligand bound specifically to the protein, wherein the first and second DNA motifs comprise domains hybridized to each other, and a third DNA motif hybridized to the first and second DNA motifs.

20. The protein-DNA complex of claim 19, further comprising a detectable beacon.

21. The protein-DNA complex of claim 20 wherein the detectable beacon comprises a fluorophore.

22. A kit for detecting a protein, comprising a providing a first nucleic acid motif linked to a first affinity ligand which binds specifically to the protein and having a first toehold domain and a first binding domain, and a second nucleic acid motif linked to a second affinity ligand which binds specifically to the protein and having a second binding domain, wherein the first and second binding domains are complementary to each other, and a displaced nucleic acid motif which is displaced by the binding of the first and second nucleic acid motifs to the protein and to each other.


SEQUENCE LISTING (36 sequences)

SEQ ID NO: 1 - DNA, 35 bp, Artificial Sequence
  Synthetic DNA motif T*C* with 6-carboxyfluorescein-labeled (FAM) at position 35
ctagagcatcacacggacacatgggatacacgctt

SEQ ID NO: 2 - DNA, 20 bp, Artificial Sequence
  Synthetic DNA motif C with dabcyl at position 1
aagcgtgtatcccatgtgtc

SEQ ID NO: 3 - DNA, 42 bp, Artificial Sequence
  Synthetic DNA motif B*C biotinylated at position 42
aagcgtgtatcccatgtgtccctcactgagactccttttttt

SEQ ID NO: 4 - DNA, 39 bp, Artificial Sequence
  Synthetic DNA motif TB T15B6 biotinylated at position 1
ttttttttttttttttgtgaggttcgtgtgatgctctag

SEQ ID NO: 5 - DNA, 33 bp, Artificial Sequence
  Synthetic DNA motif TB T9B6 biotinylated at position 1
ttttttttttttttttgtgaggttcgtgtgatg

SEQ ID NO: 6 - DNA, 32 bp, Artificial Sequence
  Synthetic DNA motif TB T8B6 biotinylated at position 1
ttttttttttttttttgtgaggttcgtgtgat

SEQ ID NO: 7 - DNA, 31 bp, Artificial Sequence
  Synthetic DNA motif TB T7B6 biotinylated at position 1
ttttttttttttttttgtgaggttcgtgtga

SEQ ID NO: 8 - DNA, 30 bp, Artificial Sequence
  Synthetic DNA motif TB T6B6 biotinylated at position 1
ttttttttttttttttgtgaggttcgtgtg

SEQ ID NO: 9 - DNA, 26 bp, Artificial Sequence
  Synthetic DNA motif T9B15
ggagtctcagtgaggttcgtgtgatg

SEQ ID NO: 10 - DNA, 28 bp, Artificial Sequence
  Synthetic DNA motif TC(T8C20)
aagcgtgtatcccatgtgtccgtgtgat

SEQ ID NO: 11 - DNA, 35 bp, Artificial Sequence
  Synthetic DNA motif Biotin-C* with 6-carboxyfluorescein-labeled (FAM) at position 35
tttttttttttttttgacacatgggatacacgctt

SEQ ID NO: 12 - DNA, 35 bp, Artificial Sequence
  Synthetic DNA motif C-Biotin
aagcgtgtatcccatgtgtcttttttttttttttt

SEQ ID NO: 13 - DNA, 55 bp, Artificial Sequence
  Synthetic DNA probe B*C for thrombin detection
aagcgtgtatcccatgtgtccctcactgagttttttttttggttggtgtggttgg

SEQ ID NO: 14 - DNA, 60 bp, Artificial Sequence
  Synthetic DNA probe TB for thrombin detection
agtccgtggtagggcaggttggggtgactttttttttttttttgtgaggttcgtgtgatg

SEQ ID NO: 15 - DNA, 35 bp, Artificial Sequence; identical to SEQ ID NO: 1
  Synthetic DNA motif T*C* used in gel electrophoresis expts

SEQ ID NO: 16 - DNA, 20 bp, Artificial Sequence
  Synthetic DNA motif C with 6-carboxyfluorescein-labeled (FAM) at position 1 used in gel electrophoresis expts
aagcgtgtatcccatgtgtc

SEQ ID NO: 17 - DNA, 27 bp, Artificial Sequence
  Synthetic DNA sequence O for binding-induced DNA strand displacement and displacement beacon
atagatcctcatagcgagacctagcaa

SEQ ID NO: 18 - DNA, 50 bp, Artificial Sequence
  Synthetic DNA sequence L for binding-induced DNA strand displacement and displacement beacon
ttagtcctacagcagtaacgactatagatcctcatagcgagacctagcaa

SEQ ID NO: 19 - DNA, 27 bp, Artificial Sequence
  Synthetic DNA sequence T(12 nt) biotinylated at position 1 for binding-induced DNA strand displacement and displacement beacon
tttttttttttttttttgctaggtctc

SEQ ID NO: 20 - DNA, 29 bp, Artificial Sequence
  Synthetic DNA sequence T(14 nt) biotinylated at position 1 for binding-induced DNA strand displacement and displacement beacon
tttttttttttttttttgctaggtctcgc

SEQ ID NO: 21 - DNA, 31 bp, Artificial Sequence
  Synthetic DNA sequence T(16 nt) biotinylated at position 1 for binding-induced DNA strand displacement and displacement beacon
tttttttttttttttttgctaggtctcgcta

SEQ ID NO: 22 - DNA, 33 bp, Artificial Sequence
  Synthetic DNA sequence T(18 nt) biotinylated at position 1 for binding-induced DNA strand displacement and displacement beacon
tttttttttttttttttgctaggtctcgctatg

SEQ ID NO: 23 - DNA, 35 bp, Artificial Sequence
  Synthetic DNA sequence T(20 nt) biotinylated at position 1 for binding-induced DNA strand displacement and displacement beacon
tttttttttttttttttgctaggtctcgctatgag

SEQ ID NO: 24 - DNA, 27 bp, Artificial Sequence
  Synthetic DNA sequence C(12 nt) biotinylated at position 27 for binding-induced DNA strand displacement and displacement beacon
gagacctagcaattttttttttttttt

SEQ ID NO: 25 - DNA, 29 bp, Artificial Sequence
  Synthetic DNA sequence C(14 nt) biotinylated at position 29 for binding-induced DNA strand displacement and displacement beacon
gcgagacctagcaattttttttttttttt

SEQ ID NO: 26 - DNA, 31 bp, Artificial Sequence
  Synthetic DNA sequence C(16 nt) biotinylated at position 31 for binding-induced DNA strand displacement and displacement beacon
tagcgagacctagcaattttttttttttttt

SEQ ID NO: 27 - DNA, 20 bp, Artificial Sequence
  Synthetic DNA sequence F with 6-carboxyfluorescein-labeled (FAM) at position 1 for binding-induced DNA strand displacement and displacement beacon
atagatcctcatagcgagac

SEQ ID NO: 28 - DNA, 27 bp, Artificial Sequence
  Synthetic DNA sequence Q with dabcyl at position 27 for binding-induced DNA strand displacement and displacement beacon
ttgctaggtctcgctatgaggatctat

SEQ ID NO: 29 - DNA, 27 bp, Artificial Sequence
  Synthetic DNA sequence O for binding-induced catalytic DNA circuit
atagatcctcatagcgagacctagcaa

SEQ ID NO: 30 - DNA, 66 bp, Artificial Sequence
  Synthetic DNA sequence H1 for binding-induced catalytic DNA circuit
ctaggtctcgctatgaggatctaccatcgtgtactagatcctcatagcgaaagagcacccttgtca

SEQ ID NO: 31 - DNA, 46 bp, Artificial Sequence
  Synthetic DNA sequence H2 for binding-induced catalytic DNA circuit
aggatctagtacacgatggtagatcctcatagcgaccatcgtgtac

SEQ ID NO: 32 - DNA, 24 bp, Artificial Sequence
  DNA sequence F′ with 6-carboxyfluorescein-labeled (FAM) at  position 1 for binding-induced catalytic DNA circuit
tgacaagggtgctctttcgctatg

SEQ ID NO: 33 - DNA, 16 bp, Artificial Sequence
  Synthetic DNA sequence Q′ with dabcyl at position 16 for binding-induced catalytic DNA circuit
aagagcacccttgtca

SEQ ID NO: 34 - DNA, 24 bp, Artificial Sequence
  Synthetic DNA sequence P for binding-induced catalytic DNA circuit
catagcgaaagagcacccttgtca

SEQ ID NO: 35 - DNA, 25 bp, Artificial Sequence
  Synthetic binding-induced DNA strand O
atagatcctcatagcgagacctagc

SEQ ID NO: 36 - DNA, 80 bp, Artificial Sequence
  Synthetic binding-induced DNA strand Apt-T(12 nt)′
tactcagggcactgcaagcaattgtggtcccaatgggctgagtattttttttttttttttttttttttttgctaggtctc